(19) United States
(12) Patent Application Publication
(10) Pub. No.: US 2024/0165093 A1
(43) Pub. Date: May 23, 2024

(54) METHODS FOR TREATING FAMILIAL CHYLOMICRONEMIA SYNDROME

(71) Applicant: AMRYT PHARMACEUTICALS INC., Boston, MA (US)

(72) Inventor: Mark SUMERAY, Zug (CH)

(21) Appl. No.: 18/548,798

(22) PCT Filed: Mar. 3, 2022
(86) PCT No.: PCT/US2022/018672

§ 371 Date: Sep. 1, 2023

Related U.S. Application Data
(60) Provisional application No. 63/165,457, filed on Mar. 24, 2021.
(60) Provisional application No. 63/155,960, filed on Mar. 3, 2021.

(51) Int. Cl. A61K 31/4468; A61K 9/00; A61P 3/06

(52) U.S. Cl. CPC A61K 31/4468; A61K 9/0053; A61P 3/06

(57) ABSTRACT

Provided herein are methods of treating familial chylomicronemia syndrome (FCS) with compositions comprising lomitapide or a pharmaceutically acceptable salt thereof.

### CROSS-REFERENCE TO RELATED APPLICATIONS

This application claims the benefit of priority to U.S. Application Ser. No. 63/165,457 filed on Mar. 24, 2021, and U.S. Application Ser. No. 63/155,960 filed on Mar. 3, 2021, the contents of each of which are hereby incorporated by reference in their entireties for all purposes.

### BACKGROUND

Familial chylomicronemia Syndrome (FCS) is a rare, genetic condition characterized by extremely high levels of plasma triglycerides and is estimated to occur in approximately 1 in 1,000,000 individuals worldwide.

The extreme triglyceride elevations in FCS lead to periodic abdominal pain, which is often seen in childhood. As the disease progresses later in life, it can result in multiple and recurrent episodes of acute pancreatitis, associated abdominal pain, xantomatosis (plain, eruptive and tuberous), lipemia retinalis, and renal failure. Occasionally, pancytopenia (due to the presence of lipid-laden macrophages in the bone marrow) and neurological symptoms such as depression and cognitive impairment have also been reported. The major cause of morbidity in FCS patients is recurrent pancreatitis leading to pancreatic insufficiency and, ultimately, pancreatic failure.

Patients with FCS fail to respond to currently available lipid-lowering agents (e.g., fibrates and omega-3 fatty acids) so dietary management with an extremely low-fat diet and supplementation with Medium Chain Tryglicerides (MCT)-remains the cornerstone of therapy. However, TG levels remain significantly elevated in most FCS patients despite severe dietary restrictions and FCS patients are exposed to acute pancreatitis.

Thus, there is an unmet need for safe and effective treatments for patients with FCS.

### SUMMARY

The present disclosure provides, among other things, methods of treating familial chylomicronemia syndrome (FCS) in a patient in need thereof, the method comprising:
a) orally administering a first daily dose of about 5 mg of lomitapide, or a pharmaceutically acceptable salt thereof, to the patient for a first dosing period;
b) measuring the fasting triglyceride levels of the patient after the first dosing period, wherein if the patient's fasting triglyceride levels are ≤1000 mg/dL while adhering to a low-fat diet, the patient is maintained at a daily dose of lomitapide, or a pharmaceutically acceptable salt thereof, of about 5 mg;
c) if the patient's measured fasting triglyceride levels are >1000 mg/dL while adhering to a low-fat diet after the first dosing period, the patient is orally administered a second daily dose of about 10 mg of lomitapide, or a pharmaceutically acceptable salt thereof, for a second dosing period;
d) measuring the fasting triglyceride levels of the patient after the second dosing period, wherein if the patient's fasting triglyceride levels are ≤1000 mg/dL while adhering to a low-fat diet, the patient is maintained at a daily dose of lomitapide, or a pharmaceutically acceptable salt thereof, of about 10 mg;
e) if the patient's measured fasting triglyceride levels are >1000 mg/dL while adhering to a low-fat diet after the second dosing period, the patient is orally administered a third daily dose of about 20 mg of lomitapide, or a pharmaceutically acceptable salt thereof, for a third dosing period.

The present disclosure provides, among other things, methods of treating familial chylomicronemia syndrome (FCS) in a patient in need thereof, the method comprising:
a) orally administering a first daily dose of about 5 mg of lomitapide, or a pharmaceutically acceptable salt thereof, to the patient for a first dosing period;
b) measuring the fasting triglyceride levels of the patient after the first dosing period, wherein if the patient's fasting triglyceride levels are ≤about 750 mg/dL while adhering to a low-fat diet, the patient is maintained at a daily dose of lomitapide, or a pharmaceutically acceptable salt thereof, of about 5 mg;
c) if the patient's measured fasting triglyceride levels are >about 750 mg/dL while adhering to a low-fat diet after the first dosing period, the patient is orally administered a second daily dose of about 10 mg of lomitapide, or a pharmaceutically acceptable salt thereof, for a second dosing period;
d) measuring the fasting triglyceride levels of the patient after the second dosing period, wherein if the patient's fasting triglyceride levels are S about 750 mg/dL while adhering to a low-fat diet, the patient is maintained at a daily dose of lomitapide, or a pharmaceutically acceptable salt thereof, of about 10 mg;
e) if the patient's measured fasting triglyceride levels are >about 750 mg/dL while adhering to a low-fat diet after the second dosing period, the patient is orally administered a third daily dose of about 20 mg of lomitapide, or a pharmaceutically acceptable salt thereof, for a third dosing period.

In some embodiments, the patient in need of FCS treatment is confirmed homozygote, compound heterozygote or double heterozygote for one or more loss-of-function mutations in genes causing FCS.

In some embodiments, the patient in need of FCS treatment has a history of pancreatitis.

In some embodiments, when the patient's liver aminotransferase (ALT/AST) levels are ≥5 times the upper limit of normal (ULN) after the first dosing period, the second dosing period or the third dosing period, the patient is withdrawn from lomitapide treatment. In some embodiments, the patient is withdrawn from treatment until ALT/AST levels are <3 times the ULN. In some embodiments, the method further comprises reducing the patient's dose to the last dose that provided patient ALT/AST levels of <3 times the ULN.

In some embodiments, when the patient's ALT/AST levels are from 3-5 times the ULN, the method comprises confirming the patient's ALT/AST levels are 3-5 times the ULN within one week of the elevated ALT/AST test result. In some embodiments, the method further comprises reducing the patient's dose to the last dose that provided patient ALT/AST levels of <3 times the ULN.

In some embodiments, methods of the present disclosure comprise adjusting the patient's daily lomitapide dose to provide fasting triglyceride levels ≤1000 mg/dL and ALT/AST levels ≤3 times the ULN.

In some embodiments, the daily dose that provides fasting triglyceride levels ≤1000 mg/dL and ALT/AST levels ≤3 times the ULN is selected from the group consisting of 5 mg, 10 mg, 15 mg, 20 mg, 25 mg, 30 mg, 35 mg, 40 mg, 45 mg, 50 mg, 55 mg, and 60 mg of lomitapide or a pharmaceutically acceptable salt thereof.

In some embodiments, the daily dose that provides fasting triglyceride levels ≤1000 mg/dL and ALT/AST levels ≤3 times the ULN is selected from the group consisting of 5 mg, 10 mg, 15 mg, 20 mg, 30 mg, 40 mg, 50 mg, and 60 mg of lomitapide or a pharmaceutically acceptable salt thereof.

In some embodiments, the daily dose that provides fasting triglyceride levels ≤1000 mg/dL and ALT/AST levels ≤3 times the ULN is selected from the group consisting of 5 mg, 10 mg, 20 mg, 30 mg, and 60 mg of lomitapide or a pharmaceutically acceptable salt thereof.

In some embodiments, the daily dose that provides fasting triglyceride levels ≤1000 mg/dL and ALT/AST levels ≤3 times the ULN is selected from the group consisting of 5 mg, 10 mg, 20 mg, 40 mg, and 60 mg of lomitapide or a pharmaceutically acceptable salt thereof.

In some embodiments, the daily dose that provides fasting triglyceride levels ≤1000 mg/dL and ALT/AST levels ≤3 times the ULN is selected from the group consisting of 5 mg, 10 mg, 15 mg, and 20 mg of lomitapide or a pharmaceutically acceptable salt thereof.

In some embodiments, the daily dose that provides fasting triglyceride levels ≤1000 mg/dL and ALT/AST levels ≤3 times the ULN is 5 mg of lomitapide or a pharmaceutically acceptable salt thereof. In some embodiments, the daily dose that provides fasting triglyceride levels ≤1000 mg/dL and ALT/AST levels ≤3 times the ULN is 10 mg of lomitapide or a pharmaceutically acceptable salt thereof. In some embodiments, the daily dose that provides fasting triglyceride levels ≤1000 mg/dL and ALT/AST levels ≤3 times the ULN is 15 mg of lomitapide or a pharmaceutically acceptable salt thereof. In some embodiments, the daily dose that provides fasting triglyceride levels ≤1000 mg/dL and ALT/AST levels ≤3 times the ULN is 20 mg of lomitapide or a pharmaceutically acceptable salt thereof. In some embodiments, the daily dose that provides fasting triglyceride levels ≤1000 mg/dL and ALT/AST levels ≤3 times the ULN is 30 mg of lomitapide or a pharmaceutically acceptable salt thereof. In some embodiments, the daily dose that provides fasting triglyceride levels ≤1000 mg/dL and ALT/AST levels ≤3 times the ULN is 40 mg of lomitapide or a pharmaceutically acceptable salt thereof. In some embodiments, the daily dose that provides fasting triglyceride levels ≤1000 mg/dL and ALT/AST levels ≤3 times the ULN is 50 mg of lomitapide or a pharmaceutically acceptable salt thereof. In some embodiments, the daily dose that provides fasting triglyceride levels ≤1000 mg/dL and ALT/AST levels ≤3 times the ULN is 60 mg of lomitapide or a pharmaceutically acceptable salt thereof.

In some embodiments, the daily dose that provides fasting triglyceride levels ≤750 mg/dL and ALT/AST levels ≤3 times the ULN is selected from the group consisting of 5 mg, 10 mg, 15 mg, 20 mg, 25 mg, 30 mg, 35 mg, 40 mg, 45 mg, 50 mg, 55 mg, and 60 mg of lomitapide or a pharmaceutically acceptable salt thereof.

In some embodiments, the daily dose that provides fasting triglyceride levels ≤750 mg/dL and ALT/AST levels ≤3 times the ULN is selected from the group consisting of 5 mg, 10 mg, 15 mg, 20 mg, 30 mg, 40 mg, 50 mg, and 60 mg of lomitapide or a pharmaceutically acceptable salt thereof.

In some embodiments, the daily dose that provides fasting triglyceride levels ≤750 mg/dL and ALT/AST levels ≤3 times the ULN is 5 mg of lomitapide or a pharmaceutically acceptable salt thereof. In some embodiments, the daily dose that provides fasting triglyceride levels ≤750 mg/dL and ALT/AST levels ≤3 times the ULN is 10 mg of lomitapide or a pharmaceutically acceptable salt thereof. In some embodiments, the daily dose that provides fasting triglyceride levels ≤750 mg/dL and ALT/AST levels ≤3 times the ULN is 15 mg of lomitapide or a pharmaceutically acceptable salt thereof. In some embodiments, the daily dose that provides fasting triglyceride levels ≤750 mg/dL and ALT/AST levels ≤3 times the ULN is 20 mg of lomitapide or a pharmaceutically acceptable salt thereof. In some embodiments, the daily dose that provides fasting triglyceride levels ≤750 mg/dL and ALT/AST levels ≤3 times the ULN is 30 mg of lomitapide or a pharmaceutically acceptable salt thereof. In some embodiments, the daily dose that provides fasting triglyceride levels ≤750 mg/dL and ALT/AST levels ≤3 times the ULN is 40 mg of lomitapide or a pharmaceutically acceptable salt thereof. In some embodiments, the daily dose that provides fasting triglyceride levels ≤750 mg/dL and ALT/AST levels ≤3 times the ULN is 50 mg of lomitapide or a pharmaceutically acceptable salt thereof. In some embodiments, the daily dose that provides fasting triglyceride levels ≤750 mg/dL and ALT/AST levels ≤3 times the ULN is 60 mg of lomitapide or a pharmaceutically acceptable salt thereof.

In some embodiments, the daily dose that provides fasting triglyceride levels ≤500 mg/dL and ALT/AST levels ≤3 times the ULN is selected from the group consisting of 5 mg, 10 mg, 15 mg, 20 mg, 25 mg, 30 mg, 35 mg, 40 mg, 45 mg, 50 mg, 55 mg, and 60 mg of lomitapide or a pharmaceutically acceptable salt thereof.

In some embodiments, the daily dose that provides fasting triglyceride levels ≤500 mg/dL and ALT/AST levels ≤3 times the ULN is selected from the group consisting of 5 mg, 10 mg, 15 mg, 20 mg, 30 mg, 40 mg, 50 mg, and 60 mg of lomitapide or a pharmaceutically acceptable salt thereof.

In some embodiments, the daily dose that provides fasting triglyceride levels ≤500 mg/dL and ALT/AST levels ≤3 times the ULN is 5 mg of lomitapide or a pharmaceutically acceptable salt thereof. In some embodiments, the daily dose that provides fasting triglyceride levels ≤500 mg/dL and ALT/AST levels ≤3 times the ULN is 10 mg of lomitapide or a pharmaceutically acceptable salt thereof. In some embodiments, the daily dose that provides fasting triglyceride levels ≤500 mg/dL and ALT/AST levels ≤3 times the ULN is 15 mg of lomitapide or a pharmaceutically acceptable salt thereof. In some embodiments, the daily dose that provides fasting triglyceride levels ≤500 mg/dL and ALT/AST levels ≤3 times the ULN is 20 mg of lomitapide or a pharmaceutically acceptable salt thereof. In some embodiments, the daily dose that provides fasting triglyceride levels ≤500 mg/dL and ALT/AST levels ≤3 times the ULN is 30 mg of lomitapide or a pharmaceutically acceptable salt thereof. In some embodiments, the daily dose that provides fasting triglyceride levels ≤500 mg/dL and ALT/AST levels ≤3 times the ULN is 40 mg of lomitapide or a pharmaceutically acceptable salt thereof. In some embodiments, the daily dose that provides fasting triglyceride levels ≤500 mg/dL and ALT/AST levels ≤3 times the ULN is 50 mg of lomitapide or a pharmaceutically acceptable salt thereof. In some embodiments, the daily dose that provides fasting triglyceride levels ≤500 mg/dL and ALT/AST levels ≤3 times the ULN is 60 mg of lomitapide or a pharmaceutically acceptable salt thereof.

In some embodiments of the methods provided herein, the lomitapide administration does not provide a clinically significant increase in hepatic fat liver during the treatment period.

In some embodiments of the methods provided herein, the lomitapide administration substantially decreases the episodes of pancreatitis compared to prior to said treatment.

In some embodiments the present disclosure provides methods of treating familial chylomicronemia syndrome (FCS) in a pediatric patient in need thereof, the method comprising:
a) orally administering a first daily dose of about 2 mg to about 5 mg of lomitapide, or a pharmaceutically acceptable salt thereof, to the patient for a first dosing period;
b) measuring the fasting triglyceride levels of the patient after the first dosing period, wherein if the patient's fasting triglyceride levels are ≤1000 mg/dL, the patient is maintained at a daily dose of lomitapide, or a pharmaceutically acceptable salt thereof, of about 2 mg to about 5 mg;
c) if the patient's measured fasting triglyceride levels are ≥1000 mg/dL after the first dosing period, the patient is orally administered a second daily dose of about 5 mg to about 10 mg of lomitapide, or a pharmaceutically acceptable salt thereof, for a second dosing period;
d) measuring the fasting triglyceride levels of the patient after the second dosing period, wherein if the patient's fasting triglyceride levels are ≤1000 mg/dL, the patient is maintained at a daily dose of lomitapide, or a pharmaceutically acceptable salt thereof, of about 5 mg to about 10 mg;
e) if the patient's measured fasting triglyceride levels are >1000 mg/dL after the second dosing period, the patient is orally administered a third daily dose of about 10 mg to about 20 mg of lomitapide, or a pharmaceutically acceptable salt thereof, for a third dosing period.

In some embodiments the present disclosure provides methods of treating familial chylomicronemia syndrome (FCS) in a pediatric patient in need thereof, the method comprising:
a) orally administering a first daily dose of about 2 mg to about 5 mg of lomitapide, or a pharmaceutically acceptable salt thereof, to the patient for a first dosing period;
b) measuring the fasting triglyceride levels of the patient after the first dosing period, wherein if the patient's fasting triglyceride levels are ≤750 mg/dL, the patient is maintained at a daily dose of lomitapide, or a pharmaceutically acceptable salt thereof, of about 2 mg to about 5 mg;
c) if the patient's measured fasting triglyceride levels are >750 mg/dL after the first dosing period, the patient is orally administered a second daily dose of about 5 mg to about 10 mg of lomitapide, or a pharmaceutically acceptable salt thereof, for a second dosing period;
d) measuring the fasting triglyceride levels of the patient after the second dosing period, wherein if the patient's fasting triglyceride levels are ≤750 mg/dL, the patient is maintained at a daily dose of lomitapide, or a pharmaceutically acceptable salt thereof, of about 5 mg to about 10 mg;
e) if the patient's measured fasting triglyceride levels are >750 mg/dL after the second dosing period, the patient is orally administered a third daily dose of about 10 mg to about 20 mg of lomitapide, or a pharmaceutically acceptable salt thereof, for a third dosing period.

In some embodiments, the pediatric patient in need of FCS treatment is administered a daily dose of 2 mg of lomitapide or a pharmaceutically acceptable salt thereof in the first dosing period, a daily dose of 5 mg of lomitapide or a pharmaceutically acceptable salt thereof in the second dosing period, and a daily dose of 10 mg of lomitapide or a pharmaceutically acceptable salt thereof in the third dosing period.

In some embodiments, the pediatric patient in need of FCS treatment is administered a daily dose of 5 mg of lomitapide, or a pharmaceutically acceptable salt thereof in the first dosing period, a daily dose of 10 mg of lomitapide, or a pharmaceutically acceptable salt thereof in the second dosing period, and a daily dose of 20 mg of lomitapide, or a pharmaceutically acceptable salt thereof in the third dosing period.

### BRIEF DESCRIPTION OF THE DRAWINGS

FIG. 1 shows mean triglyceride (TG) levels (mg/dL) in FCS patients (n=18) treated with lomitapide over 26 weeks from the study described in Example 1.

FIG. 2 shows mean total cholesterol (TC) levels (mg/dL) in FCS patients (n=18) treated with lomitapide over 26 weeks from the study described in Example 1. Terms TC or CT are used interchangeably.

FIG. 3 shows mean HDL-C levels (mg/dL) in FCS patients (n=18) treated with lomitapide over 26 weeks from the study described in Example 1.

FIG. 4 shows the mean lomitapide dose (mg) administered to FCS patients (n=18) over the course of the 26-week study described in Example 1

FIG. 5 shows mean liver ALT levels (ul/L) in FCS patients (n=18) treated with lomitapide over the course of the 26-week study described in Example 1.

FIG. 6 shows mean liver AST levels (ul/L) in FCS patients (n=18) treated with lomitapide over the course of the 26-week study described in Example 1.

FIG. 7 shows median triglyceride levels in FCS patients receiving lomitapide over the course of the 26-week study described in Example 1.

FIG. 8 shows change in triglyceride levels (%) in FCS patients receiving lomitapide from baseline to Week 26 in the study described in Example 1.

FIG. 9 shows median liver transaminase levels in FCS patients receiving lomitapide over the course of the 26-week study described in Example 1.

### DEFINITIONS

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Throughout this disclosure, various patents, patent applications and publications are referenced. The disclosures of these patents, patent applications and publications are incorporated in their entireties into this disclosure by reference in order to more fully describe the state of the art as known to those skilled therein as of the date of this disclosure. This disclosure will govern in the instance that there is any inconsistency between the patents, patent applications and publications cited and this disclosure.

The term “about” when immediately preceding a numerical value means a range of plus or minus an acceptable degree of variation in the art. In some embodiments, the term “about” encompasses 10% of that value, e.g., “about 50” means 45 to 55, “about 25,000” means 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

Throughout the present disclosure, numerical ranges are provided for certain quantities. These ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The terms “administer,” “administering” or “administration” as used herein refer to either directly administering a compound, or a salt, solvate or prodrug thereof or a composition comprising the compound, or a salt, solvate or prodrug thereof to a patient.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical agent from one organ, or portion of the body, to another organ or portion of the body.

The term “treating” as used herein with regard to a patient, refers to improving at least one symptom of the patient's disorder. Treating can be improving, or at least partially ameliorating a disorder.

The terms “effective amount” and “therapeutically effective amount” are used interchangeably in this disclosure and refer to an amount of a compound, or a salt, solvate or prodrug thereof, that, when administered to a patient, is capable of performing the intended result. For example, an effective amount of lomitapide, or a pharmaceutically acceptable salt thereof is that amount which is required to reduce at least one symptom of FCS in a patient, e.g. the amount required to reduce the frequency of pancreatitis in a patient with FCS compared to prior to treatment. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The term “therapeutic effect” as used herein refers to a desired or beneficial effect provided by the method and/or the composition. For example, the method for treating FCS provides a therapeutic effect when the method reduces at least one symptom of FCS, e.g., reduce the frequency of pancreatitis in a patient with FCS compared to prior to treatment.

The phrase “pediatric patient” as used herein refers to a patient less than 18 years of age.

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

### DETAILED DESCRIPTION

### Familial Chylomicronemia Syndrome

Familial Chylomicronemia Syndrome (FCS) is a rare recessive genetic disorder, inherited as monogenic recessive trait. The main biochemical feature of FCS is the accumulation of chylomicrons (CM) in plasma with triglyceride (TG) levels >10 mmol/L.

The extreme triglyceride elevations in FCS lead to periodic abdominal pain, which is often seen in childhood. As the disease progresses later in life, it can result in multiple and recurrent episodes of acute pancreatitis, associated abdominal pain, xanthomatosis (plain, eruptive and tuberous), lipemia retinalis, and renal failure. Acute pancreatitis is the most frequent and damaging complication of FCS, resulting in chronic malabsorption and diabetes mellitus in a subgroup of FCS patients. Episodes of pancreatitis can also be life-threatening, result in the need for intensive care treatment. The mortality rate for hypertriglyceridemia-induced acute pancreatitis has been reported to range from 5 to 30%. Plasma triglyceride levels greater than 1000 mg/dL, a key threshold for increased risk of pancreatitis, generally reflect an abundance of triglycerides carried within chylomicrons particles. Occasionally, pancytopenia (due to the presence of lipid-laden macrophages in the bone marrow) and neurological symptoms such as depression and cognitive impairment have also been reported. Other symptoms of FCS can include nausea, diarrhea, bloating, physical weakness, constipation, indigestion, fatigue, and hepatosplenomegaly.

Genetic causes of FCS include homozygotes, compound heterozygotes or double heterozygotes for loss-of-function mutations in known genes involved in peripheral hydrolysis of triglyceride rich lipoproteins (VLDL and chylomicrons): Lipoprotein Lipase (LPL), Apolipoprotein (APO) C2, APOA5, glycosylphosphatidylinositol-anchored high-density lipoprotein-binding protein 1 (GPI-HBP1), or lipase maturation factor 1 (LMF1). LPL deficiency (LPLD), due to a primary defect of LPL, LPLD is the most frequent and studied lipolytic defect responsible of FCS worldwide. More than fifty patients with LPL mutations have been described in Italy and 48% of the cases suffered of at least an episode of acute pancreatitis.

### Lomitapide

Lomitapide is a first in class oral, selective inhibitor of microsomal transfer protein (MTP), an intracellular lipid-transfer protein that is found in the lumen of the endoplasmic reticulum and is responsible for binding and shuttling individual lipid molecules between membranes. MTP plays a key role in the assembly of apo B containing lipoproteins in the liver and intestines. Inhibition of MTP reduces lipoprotein secretion and circulating concentrations of lipoprotein-borne lipids including cholesterol and triglycerides.

The chemical name of lomitapide is N-(2,2,2-trifluoroethyl)-9-[4-[4-[[[4′(trifluoromethyl) [1,1′-biphenyl]-2-yl]carbonyl]amino]-1-piperidinyl]butyl]-9H-fluorene-9carboxamide. Its structural formula is:

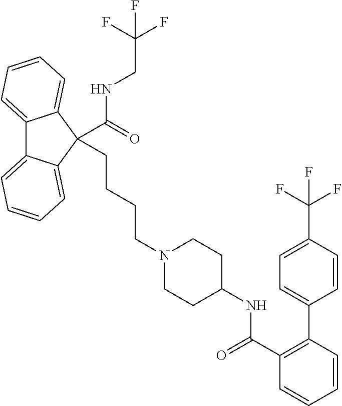 [SMILES: O=C(NC1CCN(CCCCC2(C(=O)NCC(F)(F)F)c3ccccc3-c3ccccc32)CC1)c1ccccc1-c1ccc(C(F)(F)F)cc1]

Lomitapide and other inhibitors of MTP-mediated neutral lipid transfer activity are described, for example, in U.S. Pat. Nos. 5,789,197, 5,883,109, 6,066,653, and 6,492,365, each of which is incorporated herein by reference in its entirety. MTP inhibitors are described throughout U.S. Pat. No. 6,066,653, in particular in columns 3-28. Lomitapide and methods for its use are described, for example, in U.S. Pat. Nos. 7,932,268; 8,618,135; 9,265,758; 9,364,470; 9,433,617; 9,861,622, 10,016,404, 10,555,938 each of which is incorporated by reference herein in its entirety. See also U.S. Pat. No. 10,213,419 the entirety of which is incorporated herein by reference.

The European Commission (EC) granted authorization for lomitapide under the trade name ‘Lojuxta®’ in July 2013. Lomitapide is a “lipid modifying agent” according to the Anatomical Therapeutic Chemical (ATC) Classification System (ATC code C10AX12). It is indicated as an adjunct to a low-fat diet and other lipid-lowering medicinal products with or without LA in adult patients with HoFH. Genetic confirmation of HoFH should be obtained whenever possible. Other forms of primary hyperlipoproteinaemia and secondary causes of hypercholesterolaemia (e.g., nephrotic syndrome, hypothyroidism) must be excluded.

The U.S. Food & Drug Administration (FDA) granted authorisation for lomitapide under the trade name ‘Juxtapid’ in December 2012. Juxtapid® is indicated as an adjunct to a low-fat diet and other lipid-lowering treatments, including LDL apheresis where available, to reduce low-density lipoprotein cholesterol (LDLC), total cholesterol (TC), apolipoprotein B (apo B), and non-high-density lipoprotein cholesterol (non-HDL-C) in patients with homozygous familial hypercholesterolemia (HoFH).

However, Lojuxta and Juxtapid are not approved in pediatric patients (i.e., in patients less than 18 years of age) because the safety and efficacy of lomitapide in this sensitive population has not been established.

In formulating the compositions, lomitapide or a pharmaceutically acceptable salt thereof, in the amounts described herein, are compounded according to accepted pharmaceutical practice with a physiologically acceptable vehicle, carrier, excipient, binder, preservative, stabilizer, flavor, etc., in the particular type of unit dosage form. Such dosage forms can be administered to the patient on a regimen of one to four doses per day.

In one aspect, the disclosure provides tablets containing a lomitapide composition as described herein. A tablet may be made by compression or molding, optionally with one or more accessory ingredients. Compressed tablets may be prepared using binder (for example, gelatin, microcrystalline cellulose, or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art. The disclosed excipients may serve more than one function. For example, fillers or binders may also be disintegrants, glidants, anti-adherents, lubricants, sweeteners and the like

Liquid formulations can also be prepared by dissolving or suspending one or the combination of active substances in a conventional liquid vehicle acceptable for pharmaceutical administration so as to provide the desired dosage in one to four teaspoonsfuls.

Dosage units including tablets, capsules and caplets, of various sizes can be prepared, e.g., of about 2 to 10000 mg in total weight, containing one or both of the active substances in the ranges described above, with the remainder being a physiologically acceptable carrier of other materials according to accepted pharmaceutical practice. These tablets can, of course, be scored to provide for fractional doses. Gelatin capsules can be similarly formulated. For example, in some embodiments a scored tablet may provide the dosage unit. Under the direction of a physician or other medical professional, the subject may be directed to take one portion of the dosage unit, wherein the one portion will provide the desired dosage level for given interval. At the following interval, the patient may be instructed to take two or more portions of the dosage unit wherein the two or more portions will provide the desired dosage level for that interval.

Formulations of lomitapide are commercially available, for example, as Juxtapid capsules. Each Juxtapid capsule contains lomitapide mesylate equivalent to 5, 10, 20, or 30 mg lomitapide free base and the following inactive ingredients: pregelatinized starch, sodium starch glycolate, microcrystalline cellulose, lactose monohydrate, silicon dioxide and magnesium stearate. The capsule shells of all strengths contain gelatin and titanium dioxide; the 5 mg, 10 mg and 30 mg capsules also contain red iron oxide; and the 30 mg capsules also contain yellow iron oxide. The imprinting ink contains shellac, black iron oxide, and propylene glycol. However, the scope of the present disclosure is not limited to dosage strengths of Juxtapid presently available commercially, and includes capsules containing lomitapide mesylate (or other pharmaceutically acceptable salts of lomitapide) equivalent to 5, 10, 20, 30, 40, or 60 mg lomitapide free base.

### Methods of the Present Disclosure

In one aspect, the present disclosure provides a method of treating familial chylomicronemia syndrome (FCS) in a patient in need thereof by administering an effective amount of lomitapide, or a pharmaceutically acceptable salt thereof (e.g., lomitapide mesylate). In some embodiments, lomitapide or a pharmaceutically acceptable salt thereof is used to treat a patient with FCS, to treat FCS, to treat the symptoms of FCS, to control genetic hypertriglyceridemia in a patient with FCS, or substantially decrease the episodes of pancreatitis in a patient with FCS compared to prior to treatment. In some embodiments of the methods of treating FCS disclosed herein, administering an effective amount of lomitapide, or a pharmaceutically acceptable salt thereof controls symptoms associated with hypertriglyceridemia in a patient with FCS. In embodiments, administering an effective amount of lomitapide, or a pharmaceutically acceptable salt thereof according to any of the methods of the present disclosure, substantially decreases the episodes of pancreatitis in a patient with FCS compared to prior to treatment.

It will be understood that in some embodiments of the methods provided herein, the patient is a human. In some embodiments the patient is an adult patient. In some embodiments, the patient is a pediatric patient. In some embodiments, the patient has a history of pancreatitis (e.g., acute pancreatitis). In some embodiments, the patient's post-heparin plasma lipoprotein lipase (LpL) activity is ≤20% of normal. In some embodiments, the patient has confirmed presence of LpL inactivating antibodies.

In some embodiments, the patient has fasting triglyceride levels >1000 mg/dL prior to treatment with lomitapide or a pharmaceutically acceptable salt thereof. In some embodiments, the patient has fasting fasting triglyceride levels >750 mg/dL prior to treatment with lomitapide or a pharmaceutically acceptable salt thereof.

In some embodiments, the patient's FCS is refractory to previous treatment regimens. In some embodiments, the patient's FCS is refractory to plasma LDL apheresis. In some embodiments, the FCS patient has genetic hypertriglyceridemia and recurrent acute pancreatitis and is refractory to previous treatment regimens.

In some embodiments, the patient is a confirmed homozygote, compound heterozygote or double heterozygote for one or more loss-of-function mutations in genes causing FCS. In some embodiments, the patient has a loss-of-function mutations in genes involved in peripheral hydrolysis of triglyceride rich lipoproteins (VLDL and chylomicrons). In embodiments, the patient has a mutation in one or more genes independently selected from a gene encoding (LPL), apolipoprotein (APO) C2, APOA5, glycosylphosphatidylinositol-anchored high-density lipoprotein-binding protein 1 (GPI-HBP1), or lipase maturation factor 1 (LMF1). In some embodiments, the patient has a mutation in the gene encoding lipoprotein lipase (LPL), apolipoprotein (APO) C2, APOA5, glycosylphosphatidylinositol-anchored high-density lipoprotein-binding protein 1 (GPI-HBP1), or lipase maturation factor 1 (LMF1).

In some embodiments, the patient has a mutation in the gene encoding lipoprotein lipase (LPL). In some embodiments, the patient has a mutation in the gene encoding apolipoprotein (APO) C2. In some embodiments, the patient has a mutation in the gene encoding APOA5. In some embodiments, the patient has a mutation in the gene encoding glycosylphosphatidylinositol-anchored high-density lipoprotein-binding protein 1 (GPI-HBP1). In some embodiments, the patient has a mutation in the gene encoding lipase maturation factor 1 (LMF1). In some embodiments, the patient has a lipoprotein lipase deficiency (LPLD). In some embodiments the patient has a mutation in the gene encoding lipoprotein lipase. In some embodiments, the patient has a mutation in the gene encoding LPL and a mutation in the gene encoding APOA5. In some embodiments, the patient has a mutation in the gene encoding LPL and a mutation in the gene encoding GPIHBPI.

In some embodiments of the methods disclosed herein, the patient has one or more mutations selected from: c.250-1 G>C (IVS2); c.829 G>A (p.Asp277Asn); c.1174 C>G, (p.Leu392Val)/c.457G>A (p.Val153Met); c.984G>T (p.Met328Ile)/c.41G>T (p.Cys14Phe); c.1019-2A>T (IVS6); c.832_833delTC (p.Asp277Asp fsX4); c.987C>A (p.Tyr329Ter); c.651delT (p.Pro217Pro Fs34X); c.326T>C (p.Ile109Thr); c.644G>A (p.Gly215Glu); c.1019-2A>T (IVS6); c.621C>G (p.Asp207Glu); c.542G>A (p.Gly181Asp); c.755C>T (p.Ile252Thr) c.(?_-1)_(*1_?)del; c.621C>G (p.Asp207Glu); c.1174C>G (p.Leu392Val); c.177 C>A p.Tyr59Ter; or c.274C>T (p.Gln92Ter).

In some embodiments, the patient has the mutation c.250-1 G>C (IVS2).

In some embodiments, the patient has the mutation c.829 G>A (p.Asp277Asn).

In some embodiments, the patient has the mutation c.1174 C>G, (p.Leu392Val)/c.457G>A (p.Val153Met).

In some embodiments, the patient has the mutation c.984G>T (p.Met328Ile)/c.41G>T (p.Cys14Phe).

In some embodiments, the patient has the mutation c.1019-2A>T (IVS6).

In some embodiments, the patient has the mutation c.832_833delTC (p.Asp277Asp fsX4).

In some embodiments, the patient has the mutation c.987C>A (p.Tyr329Ter).

In some embodiments, the patient has the mutation c.651delT (p.Pro217Pro Fs34X).

In some embodiments, the patient has the mutation c.326T>C (p.Ile109Thr).

In some embodiments, the patient has the mutation c.644G>A (p.Gly215Glu).

In some embodiments, the patient has the mutation c.1019-2A>T (IVS6).

In some embodiments, the patient has the mutation c.621C>G (p.Asp207Glu).

In some embodiments, the patient has the mutation c.542G>A (p.Gly181Asp).

In some embodiments, the patient has the mutation c.755C>T (p.Ile252Thr) c.(?_-1)_(*1_?)del; c.621C>G (p.Asp207Glu).

In some embodiments, the patient has the mutation c.1174C>G (p.Leu392Val).

In some embodiments, the patient has the mutation c.177 C>A p.Tyr59Ter.

In some embodiments, the patient has the mutation or c.274C>T (p.Gln92Ter.

In some embodiments of the methods disclosed herein, the patient has one or more mutations in the LPL gene selected from c.250-1 G>C (IVS2); c.829 G>A (p.Asp277Asn); c.1174 C>G, (p.Leu392Val); c.984G>T (p.Met328Ile); c.1019-2A>T (IVS6); c.832_833delTC (p.Asp277Asp fsX4); c.987C>A (p.Tyr329Ter); c.651delT (p.Pro217Pro Fs34X); c.326T>C (p.Ile109Thr); c.644G>A (p.Gly215Glu); c.1019-2A>T (IVS6); c.621C>G (p.Asp207Glu); c.542G>A (p.Gly181Asp); c.755C>T (p.Ile252Thr) c.(?_-1)_(*1_?)del; c.621C>G (p.Asp207Glu); or c.1174C>G (p.Leu392Val).

In some embodiments of the methods disclosed herein, the patient has one or more mutations in the APOC2 gene selected from c.177 C>A p.Tyr59Ter; or c.274C>T (p.Gln92Ter).

In some embodiments of the methods disclosed herein, the patient expresses a microsomal triglyceride transport protein gene (MTP) variant that improves the patient's response to lomitapide treatment compared to patients that do not express the MTP variant.

In some embodiments of the methods disclosed herein, lomitapide is administered as an adjunct to a low-fat diet and other lipid-lowering treatments (such as, statin, ezetimibe, nicotinic acid, bile acid sequestrant, fibrates (e.g., fenofibrate) or LDL apheresis, or combinations thereof). In some embodiments, lomitapide is administered as an adjunct to a low-fat diet and omega-3 fatty acids. In some embodiments, the low-fat diet comprises a diet wherein less than about 30% of patient's total calories are from fat, less than about 20% of patient's total calories are from fat, less than about 15% of patient's total calories are from fat, or less than about 10% of patient's total calories are from fat. In some embodiments, the low-fat diet comprises a diet wherein less than about 20% of patient's total calories are from fat. In some embodiments, the low-fat diet comprises a diet wherein less than about 10% of patient's total calories are from fat.

In some embodiments, patients receiving lipid lowering therapies during treatment with lomitapide are administered dietary supplements that provided approximately 400 international units vitamin E, 210 mg alpha-linolenic acid (ALA), 200 mg linoleic acid, 110 mg eicosapentaenoic acid (EPA), and 80 mg docosahexaenoic acid (DHA) per day.

In some embodiments of the present disclosure, the method of treating FCS provides a reduction in fasting triglyceride levels. For example, in accordance with some embodiments of the present disclosure, the method of treating FCS provides about a 5-95% reduction in the patients fasting triglyceride levels when compared to baseline after a specified period of time or when compared to placebo or lack of treatment, including about a 5% reduction, about a 10% reduction, about a 15% reduction, about a 20% reduction, about a 25% reduction, about a 30% reduction, about a 35% reduction, about a 40% reduction, about a 45% reduction, about a 50% reduction, about a 55% reduction, about a 60% reduction, about a 65% reduction, about a 70% reduction, about a 75% reduction, about a 80% reduction, about a 85% reduction, about a 90% reduction, about a 95% reduction or more, (including any subrange or value therebetween) in the patients fasting triglyceride levels when compared to baseline after a specified period of time or when compared to placebo or lack of treatment. In some embodiments, administering lomitapide provides a reduction in fasting triglyceride levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% when compared to baseline after a specified period of time or when compared to placebo or lack of treatment. In some embodiments, the specified period of time is about or at least about two weeks, about or at least about four weeks, about or at least about six weeks, about or at least about 10 weeks, about or at least about 14 weeks, about or at least about 18 weeks, about or at least about 22 weeks, about or at least about 26 weeks, about or at least about 28 weeks, or about or at least about 30 weeks. In some embodiments, the specified period of time is about or at least about 26 weeks.

In some embodiments, administering lomitapide provides a reduction in fasting triglyceride levels of greater than or equal to 20% compared to baseline prior to treatment. In some embodiments, administering lomitapide provides a reduction in fasting triglyceride levels of greater than or equal to 30% compared to baseline prior to treatment. In some embodiments, administering lomitapide provides a reduction in fasting triglyceride levels of greater than or equal to 40% compared to baseline prior to treatment. In some embodiments, administering lomitapide provides a reduction in fasting triglyceride levels of greater than or equal to 50% compared to baseline prior to treatment. In some embodiments, administering lomitapide provides a reduction in fasting triglyceride levels of greater than or equal to 60% compared to baseline prior to treatment. In some embodiments, administering lomitapide provides a reduction in fasting triglyceride levels of greater than or equal to 70% compared to baseline prior to treatment. In some embodiments, administering lomitapide provides a reduction in fasting triglyceride levels of greater than or equal to 80%, or more compared to baseline prior to treatment.

In some embodiments of the present disclosure, the methods of treating FCS provide a fasting triglyceride level in the range of about 10 mg/dL to about 1000 mg/dL in the patient, including about 10 mg/dL, about 20 mg/dL, about 30 mg/dL, about 40 mg/dL, about 50 mg/dL, about 60 mg/dL, about 70 mg/dL, about 80 mg/dL, about 90 mg/dL, about 100 mg/dL, about 150 mg/dL, mg/dL, about 200 mg/dL, about 250 mg/dL, about 300 mg/dL, about 350 mg/dL, about 400 mg/dL, about 450 mg/dL, about 500 mg/dL, about 550 mg/dL, about 600 mg/dL, about 650 mg/dL, about 700 mg/dL, about 750 mg/dL, about 800 mg/dL, about 850 mg/dL, about 900 mg/dL, about 950 mg/dL, or about 1000 mg/dL, including any subrange or value therebetween. In embodiments, the methods of treating FCS provide a fasting triglyceride level of about 100 mg/dL to about 900 mg/dL, or about 200 mg/dL to about 800 mg/dL in the patient.

In some embodiments of the present disclosure, the method of treating FCS provides a fasting triglyceride level of ≤1000 mg/dL, ≤950 mg/dL, ≤900 mg/dL, ≤850 mg/dL, ≤800 mg/dL, ≤750 mg/dL, ≤700 mg/dL, ≤650 mg/dL, ≤600 mg/dL, ≤550 mg/dL, ≤500 mg/dL, ≤450 mg/dL, ≤400 mg/dL, ≤350 mg/dL, ≤300 mg/dL, ≤250 mg/dL, ≤200 mg/dL, or ≤150 mg/dL. In some embodiments of the present disclosure, the method of treating FCS provides a reduction in fasting triglyceride levels to ≤about 1000 mg/dL. In some embodiments of the present disclosure, the method of treating FCS provides a reduction in fasting triglyceride levels to ≤about 750 mg/dL. In some embodiments of the present disclosure, the method of treating FCS provides a reduction in fasting triglyceride levels to ≤about 500 mg/dL.

In some embodiments of the present disclosure, the method of treating FCS provides fasting triglyceride levels ≤1000 mg/dL and ALT/AST levels ≤5 times the ULN.

In some embodiments of the present disclosure, the method of treating FCS provides fasting triglyceride levels ≤about 750 mg/dL and ALT/AST levels ≤5 times the ULN.

In some embodiments of the present disclosure, the method of treating FCS provides fasting triglyceride levels ≤about 500 mg/dL and ALT/AST levels ≤5 times the ULN.

In some embodiments of the present disclosure, the method of treating FCS provides fasting triglyceride levels ≤1000 mg/dL and ALT/AST levels ≤3 times the ULN.

In some embodiments of the present disclosure, the method of treating FCS provides fasting triglyceride levels ≤about 750 mg/dL and ALT/AST levels ≤3 times the ULN.

In some embodiments of the present disclosure, the method of treating FCS provides fasting triglyceride levels ≤about 500 mg/dL and ALT/AST levels ≤3 times the ULN.

In some embodiments, the patient's change in hepatic fat liver is measured during the treatment period. In some embodiments, the administration of lomitapide or a pharmaceutically acceptable salt thereof provides therapeutic treatment of FCS and does not provide a clinically significant increase in hepatic fat liver during the treatment period.

In some embodiments of the methods of treating FCS, the administration of lomitapide or a pharmaceutically acceptable salt thereof provides therapeutic treatment of FCS and substantially decreases the episodes of pancreatitis compared to prior to treatment.

In some embodiments of the methods of treating FCS, the administration of lomitapide or a pharmaceutically acceptable salt thereof provides therapeutic treatment of FCS and prevents episodes of pancreatitis.

In some embodiments of the methods disclosed herein, the administration of lomitapide or a pharmaceutically acceptable salt thereof provides therapeutic treatment of FCS with no significant changes in non-invasive liver fibrosis measurements such as quantification of liver stiffness, fibrosis-4 index (FIB-4) and NFS scores.

In some embodiments of the methods of disclosed herein, the administration of lomitapide or a pharmaceutically acceptable salt thereof is therapeutically effective and tolerated (e.g., treatment emergent gastrointestinal events if present, are mild or moderate (e.g., diarrhea, bloody diarrhea, nausea, dyspepsia, vomiting, abdominal pain, constipation, meteorism, and/or flatulence). In some embodiments of the methods disclosed herein, the administration of an effective amount of lomitapide or a pharmaceutically acceptable salt thereof provides therapeutic treatment of FCS, and is safe and tolerated (e.g., ALT/AST levels ≤3 times the ULN and/or gastrointestinal side effects, if present, are mild or moderate).

The FIB-4 score is a non-invasive liver fibrosis assessment tool. For example, a score ≤1.45 has a negative predictive value of over 90% for advanced liver fibrosis of multiple aetiologies and a score of >3.25 has a positive predictive value of 65% for advanced fibrosis with a specificity of 97%.

In some embodiments of the methods of treating FCS, the patient administered lomitapide or a pharmaceutically acceptable salt thereof has a FIB-4 score of less than about 1.45. In embodiments, of the methods of treating FCS, the patient administered lomitapide or a pharmaceutically acceptable salt thereof has a FIB-4 score of less than about 3.25.

NAFLD fibrosis score (NFS) is a non-invasive liver fibrosis scoring system described e.g., in Angulo P, Hui J M, Marchesini G, et al. The NAFLD fibrosis score: a noninvasive system that identifies liver fibrosis in patients with NAFLD. Hepatology 2007; 45(4):846-54, which is hereby incorporated by reference herein. A NFS<−1.455=no significant fibrosis (stage 0-2). A NFS−1.455−0.675=indeterminate score. A NFS>0.675=significant fibrosis (stage 3-4). In embodiments, the patient administered lomitapide or a pharmaceutically acceptable salt thereof has a NFS of less than about 0.675. In embodiments, the patient administered lomitapide or a pharmaceutically acceptable salt thereof has a NFS of less than about 0.5, 0.4, 0.4, 0.2 or 0.1. In embodiments, the patient administered lomitapide or a pharmaceutically acceptable salt thereof has a NFS of less than about −1.455.

According to some embodiments of the present disclosure, the method of treating FCS provides statistically significant therapeutic effect. In one embodiment, the statistically significant therapeutic effect is determined based on one or more standards or criteria provided by one or more regulatory agencies in the United States, e.g., FDA or other countries. In another embodiment, the statistically significant therapeutic effect is determined based on results obtained from regulatory agency approved clinical trial set up and/or procedure.

### Dosing

The disclosure provides methods for treating FCS by administering an effective and tolerable amount of lomitapide or a pharmaceutically acceptable salt thereof, to a patient (e.g., adult or pediatric patient) in need thereof. An effective amount is an amount sufficient to eliminate or significantly reduce FCS symptoms or to alleviate those symptoms (e.g., reduce the frequency of pancreatitis in a patient with FCS compared to prior to treatment). Alternatively, an effective amount is an amount sufficient to significantly reduce triglyceride levels in a patient, for example to fasting triglyceride levels ≤1000 mg/dL as described herein. Formulations employed in the present methods can incorporate lomitapide or a pharmaceutically acceptable salt thereof into a formulation such that the formulation provides therapeutically effective blood plasma levels of lomitapide or a pharmaceutically acceptable salt thereof for the treatment of FCS.

In some embodiments, a therapeutically effective dose is achieved by starting the patient on an initial daily dose and titrating to an efficacious and tolerated dose by gradually modifying (e.g., increasing or decreasing) the daily administered amount of lomitapide or a pharmaceutically acceptable salt thereof until a dose that is effective (i.e., the patient with FCS is treated) and tolerated is achieved. In some embodiments, the efficacious dose is a dose that improves at least one symptom of the patient's FCS. In some embodiments the dose that is effective and tolerated is a dose that provides fasting triglyceride levels ≤1000 mg/dL and ALT/AST levels ≤3 times the ULN. In some embodiments the dose that is effective and tolerated is a dose that provides fasting triglyceride levels ≤750 mg/dL and ALT/AST levels ≤3 times the ULN.

In some embodiments, the methods of the present disclosure comprise adjusting the patient's (e.g., adult patient or pediatric patient's) daily dose of lomitapide or a pharmaceutically acceptable salt thereof to provide fasting triglyceride levels ≤1000 mg/dL and ALT/AST levels ≤3 times the ULN. In some embodiments, the patient's daily dose of lomitapide or a pharmaceutically acceptable salt thereof is increased or decreased from every 2-8 weeks (e.g., every 2-4 weeks) to provide fasting triglyceride levels ≤1000 mg/dL and ALT/AST levels ≤3 times the ULN.

In some embodiments, the dose that provides fasting triglyceride levels ≤1000 mg/dL and ALT/AST levels ≤3 times the ULN is administered (e.g., once daily) for at least 2 weeks, at least 4 weeks, at least 6 weeks, at least 8 weeks, at least 10 weeks, at least 12 weeks, at least 14 weeks, at least 16 weeks, at least 18 weeks, at least 20 weeks, at least 22 weeks, at least 24 weeks, at least 26 weeks, at least 28 weeks, or at least 30 weeks. In some embodiments, the daily dose that provides fasting triglyceride levels ≤1000 mg/dL and ALT/AST levels ≤3 times the ULN is administered for at least 8 weeks.

In some embodiments, the methods of the present disclosure comprise adjusting the patient's (e.g., adult patient or pediatric patient's) daily dose of lomitapide or a pharmaceutically acceptable salt thereof to provide fasting triglyceride levels ≤750 mg/dL and ALT/AST levels ≤3 times the ULN. In some embodiments, the patient's daily dose of lomitapide or a pharmaceutically acceptable salt thereof is increased or decreased from every 2-8 weeks (e.g., every 2-4 weeks) to provide fasting triglyceride levels ≤750 mg/dL and ALT/AST levels ≤3 times the ULN.

In some embodiments, the dose that provides fasting triglyceride levels ≤750 mg/dL and ALT/AST levels ≤3 times the ULN is administered (e.g., once daily) for at least 2 weeks, at least 4 weeks, at least 6 weeks, at least 8 weeks, at least 10 weeks, at least 12 weeks, at least 14 weeks, at least 16 weeks, at least 18 weeks, at least 20 weeks, at least 22 weeks, at least 24 weeks, at least 26 weeks, at least 28 weeks, or at least 30 weeks. In some embodiments, the daily dose that provides fasting triglyceride levels ≤750 mg/dL and ALT/AST levels ≤3 times the ULN is administered for at least 8 weeks.

In some embodiments, the lomitapide or a pharmaceutically acceptable salt thereof is administered in escalating doses. In some embodiments, the escalating doses comprise at least a first dose level and a second dose level. In some embodiments, the escalating doses comprise at least a first dose level, a second dose level, and a third dose level. In some embodiments, the escalating doses further comprise a fourth dose level. In some embodiments, the escalating doses comprise a first dose level, a second dose level, a third dose level, a fourth dose level and a fifth dose level. In some embodiments, six, seven, eight, nine and ten dose levels are contemplated. In some embodiments, the patient's liver aminotransferase levels (ALT, AST) are measured prior to each dose escalation.

In some embodiments, each dose level is no more than 50% of the immediately following dose level. In some embodiments, each dose level is no more than 33% of the immediately following dose level. In some embodiments, each dose level is no more than 20% of the immediately following dose level. In some embodiments, dose levels are separated by 12 log units. In some embodiments, dose levels are separated by 1 log unit.

In some embodiments, each dose level (e.g., the first, second, third, fourth dose levels) is each independently administered to the subject for from about 2 days to about 6 months in duration. In some embodiments each dose level (e.g., the first, second, third, fourth dose levels) is each independently administered to the subject for from about or at least about 7 days to about or at least about 35 days in duration. In some embodiments each dose level (e.g., the first, second, third, fourth dose levels) is each independently administered to the subject for from about 2 weeks to about 4 weeks. In some embodiments each dose level (e.g., first, second, third, fourth dose levels) is each independently administered to the subject for about 4 weeks. In some embodiments the first, second, third dose levels are administered to the subject for from about 2 days to about 40 days and the fourth dose level is administered to the subject for from about 2 days to about or at least about 6 months.

In some embodiments, if the patient's (e.g., adult or pediatric patient) liver aminotransferase (ALT/AST) levels are ≥5 times the upper limit of normal (ULN) after a dosing period (e.g., the first dosing period, the second dosing period or the third dosing period), the patient is withdrawn from lomitapide treatment. In some embodiments, if the patient's liver aminotransferase (ALT/AST) levels are ≥5 times the upper limit of normal (ULN) the method further comprises determining the patient's alkaline phosphatase, total bilirubin and INR. In some embodiments, if the patient's liver aminotransferase (ALT/AST) levels are ≥5 times the upper limit of normal (ULN) the patient's dose is reduced to the last dose that provided patient ALT/AST levels of <3 times the ULN.

In some embodiments, if the patient's (e.g., adult or pediatric patient's) ALT/AST levels are from 3-5 times the ULN, the method comprises confirming the patient's ALT/AST levels are 3-5 times the ULN within one week of the elevated ALT/AST test result. If the elevated ALT/AST test result is confirmed, the method further comprises determining the patient's alkaline phosphatase, total bilirubin and INR. In some embodiments, the patient's ALT/AST levels, alkaline phosphatase, total bilirubin and INR are tested weekly. In some embodiments, if the patient's total bilirubin and INR increase, ALT/AST levels increase to >5 times ULN or the patient's ALT/AST levels do not fall below <3 times ULN within about 4 weeks, the patient is withdrawn from lomitapide treatment and, in some embodiments, the patient's dose is reduced to the last dose that provided patient ALT/AST levels of <3 times the ULN. In some embodiments, the dose of lomitapide or a pharmaceutically acceptable salt thereof is reduced by no more than 50% of the immediately preceding dose level, or no more than 33% of the immediately preceding dose level, or no more than 20% of the immediately preceding dose level

In some embodiments, if the patient's (e.g., adult or pediatric patient's) ALT/AST levels are from 3-5 times the ULN, or >100 U/L but <200 U/L above the baseline value, after a dosing period (e.g., after the first dosing period, the second dosing period or the third dosing period) the method further comprises reducing the patient's dose of lomitapide or a pharmaceutically acceptable salt thereof to the last dose that provided patient ALT/AST levels of <3 times the ULN. In some embodiments, the dose of lomitapide or a pharmaceutically acceptable salt thereof is reduced by no more than 50% of the immediately preceding dose level, or no more than 33% of the immediately preceding dose level, or no more than 20% of the immediately preceding dose level In some embodiments, reducing the patient's (e.g., adult or pediatric patient) dose to achieve ALT/AST levels of <3 times the ULN comprises decreasing the dose of lomitapide or a pharmaceutically acceptable salt thereof by about 5-30 mg, including about 5 mg, about 10 mg, about 15 mg, about 20 mg, or about 30 mg. In some embodiments, the dose is decreased by about 5-15 mg, or by about 5-20 mg.

In some embodiments each dose level of lomitapide or a pharmaceutically acceptable salt thereof is administered to the subject for from 2 days to 26 weeks, or more. In some embodiments each dose level is administered to the subject for from about 1 week to about 26 weeks. In some embodiments each dose level is administered to the subject for from about 1 week to about 12 weeks. In some embodiments, each dose level is administered to the subject for about 1 week to about 5 weeks. In some embodiments each dose level is administered to the subject from about 1 to about 4 weeks. In some embodiments each dose level is administered to the subject from about 1 to about 2 weeks. In some embodiments each dose level is administered to the subject from about 1 to about 2 weeks.

In some embodiments, once the patient reaches a dose that is that is effective (i.e., the patient with FCS is treated) and tolerable, the patient is maintained on the dose as long as the patient is suffering from FCS and the clinical symptoms of FCS are adequately controlled or response level is maintained. Thus the duration of treatment may be unlimited. In embodiments the duration of treatment may be for at least 6-months, one year, two years, three years, four years, five years, 6 years, 7 years, 8 years, 9 years, 10 years or more.

In some embodiments lomitapide or a pharmaceutically acceptable salt is administered. In some embodiments, lomitapide mesylate is administered. In some embodiments, lomitapide or a pharmaceutically acceptable salt thereof is administered orally.

In some embodiments, lomitapide or a pharmaceutically acceptable salt thereof is administered in a daily dose of about 0.5 mg to about 100 mg to a patient (e.g., adult or pediatric patient) with FCS in need thereof, including about 0.5 mg, about 1 mg, about 2 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg to about 100 mg, including any subrange or value therebetween. In some embodiments, about 5 mg to about 60 mg of lomitapide or a pharmaceutically acceptable salt thereof is administered on a daily basis, including about 5 mg, about 10 mg, about 20 mg, about 30 mg, about 40 mg, to about 60 mg, including any subrange or value therebetween. In some embodiments, about 5 mg, about 10 mg, about 15 mg, about 20 mg, about 30 mg, about 40 mg, about 50 mg or about 60 mg of lomitapide or a pharmaceutically acceptable salt thereof is administered on a daily basis. In some embodiments, about 5 mg of lomitapide or a pharmaceutically acceptable salt thereof is administered on a daily basis. In some embodiments, about 10 mg of lomitapide or a pharmaceutically acceptable salt thereof is administered on a daily basis. In some embodiments, about 15 mg of lomitapide or a pharmaceutically acceptable salt thereof is administered on a daily basis. In some embodiments, about 20 mg of lomitapide or a pharmaceutically acceptable salt thereof is administered on a daily basis. In some embodiments, about 30 mg of lomitapide or a pharmaceutically acceptable salt thereof is administered on a daily basis. In some embodiments, about 40 mg of lomitapide or a pharmaceutically acceptable salt thereof is administered on a daily basis. In some embodiments, about 50 mg of lomitapide or a pharmaceutically acceptable salt thereof is administered on a daily basis. In some embodiments, about 60 mg of lomitapide or a pharmaceutically acceptable salt thereof is administered on a daily basis.

In some embodiments the daily dose is administered as a single dose or divided into 2 or 3 equal or unequal doses. In some embodiments, the lomitapide is administered once-daily at bedtime.

In some embodiments, lomitapide mesylate in an amount equivalent to about 5-60 mg of the lomitapide free base is administered. In some embodiments, lomitapide mesylate in an amount equivalent to about 5 mg of the lomitapide free base is administered. In some embodiments, lomitapide mesylate in an amount equivalent to about 10 mg of the lomitapide free base is administered. In some embodiments, lomitapide mesylate in an amount equivalent to about 30 mg of the lomitapide free base is administered. In some embodiments, lomitapide mesylate in an amount equivalent to about 40 mg of the lomitapide free base is administered. In some embodiments, lomitapide mesylate in an amount equivalent to about 60 mg of the lomitapide free base is administered.

The dose administered may be adjusted according to age, weight and condition of the patient, as well as the route of administration, dosage form and regimen and the desired result.

### Adult Patients

In embodiments, the present disclosure provides methods of treating familial chylomicronemia syndrome (FCS) in a patient in need thereof, the method comprising:
a) orally administering a first daily dose of about 5 mg of lomitapide, or a pharmaceutically acceptable salt thereof, to the patient for a first dosing period;
b) measuring the fasting triglyceride levels of the patient after the first dosing period, wherein if the patient's fasting triglyceride levels are ≤1000 mg/dL while adhering to a low-fat diet, the patient is maintained at a daily dose of lomitapide, or a pharmaceutically acceptable salt thereof, of about 5 mg;
c) if the patient's measured fasting triglyceride levels are >1000 mg/dL while adhering to a low-fat diet after the first dosing period, the patient is orally administered a second daily dose of about 10 mg of lomitapide, or a pharmaceutically acceptable salt thereof, for a second dosing period.

In some embodiments, the present disclosure provides methods of treating familial chylomicronemia syndrome (FCS) in a patient in need thereof, the method comprising:
a) orally administering a first daily dose of about 5 mg of lomitapide, or a pharmaceutically acceptable salt thereof, to the patient for a first dosing period;
b) measuring the fasting triglyceride levels of the patient after the first dosing period, wherein if the patient's fasting triglyceride levels are ≤1000 mg/dL while adhering to a low-fat diet, the patient is maintained at a daily dose of lomitapide, or a pharmaceutically acceptable salt thereof, of about 5 mg;
c) if the patient's measured fasting triglyceride levels are >1000 mg/dL while adhering to a low-fat diet after the first dosing period, the patient is orally administered a second daily dose of about 10 mg of lomitapide, or a pharmaceutically acceptable salt thereof, for a second dosing period;
d) measuring the fasting triglyceride levels of the patient after the second dosing period, wherein if the patient's fasting triglyceride levels are ≤1000 mg/dL while adhering to a low-fat diet, the patient is maintained at a daily dose of lomitapide, or a pharmaceutically acceptable salt thereof, of about 10 mg;
e) if the patient's measured fasting triglyceride levels are >1000 mg/dL while adhering to a low-fat diet after the second dosing period, the patient is orally administered a third daily dose of about 15 mg to about 20 mg of lomitapide, or a pharmaceutically acceptable salt thereof, for a third dosing period.

In some embodiments, methods of the present disclosure are used to treat familial chylomicronemia syndrome (FCS) in a patient in need thereof, the method comprising:
a) orally administering a first daily dose of about 5 mg of lomitapide, or a pharmaceutically acceptable salt thereof, to the patient for a first dosing period;
b) measuring the fasting triglyceride levels of the patient after the first dosing period, wherein if the patient's fasting triglyceride levels are ≤1000 mg/dL while adhering to a low-fat diet, the patient is maintained at a daily dose of lomitapide, or a pharmaceutically acceptable salt thereof, of about 5 mg;
c) if the patient's measured fasting triglyceride levels are >1000 mg/dL while adhering to a low-fat diet after the first dosing period, the patient is orally administered a second daily dose of about 10 mg of lomitapide, or a pharmaceutically acceptable salt thereof, for a second dosing period;
d) measuring the fasting triglyceride levels of the patient after the second dosing period, wherein if the patient's fasting triglyceride levels are ≤1000 mg/dL while adhering to a low-fat diet, the patient is maintained at a daily dose of lomitapide, or a pharmaceutically acceptable salt thereof, of about 10 mg;
e) if the patient's measured fasting triglyceride levels are >1000 mg/dL while adhering to a low-fat diet after the second dosing period, the patient is orally administered a third daily dose of about 20 mg of lomitapide, or a pharmaceutically acceptable salt thereof, for a third dosing period.

In some embodiments, methods of the present disclosure are used to treat familial chylomicronemia syndrome (FCS) in a patient in need thereof, the method comprising:
a) orally administering a first daily dose of about 5 mg of lomitapide, or a pharmaceutically acceptable salt thereof, to the patient for a first dosing period;
b) measuring the fasting triglyceride levels of the patient after the first dosing period, wherein if the patient's fasting triglyceride levels are ≤1000 mg/dL while adhering to a low-fat diet, the patient is maintained at a daily dose of lomitapide, or a pharmaceutically acceptable salt thereof, of about 5 mg;
c) if the patient's measured fasting triglyceride levels are >1000 mg/dL while adhering to a low-fat diet after the first dosing period, the patient is orally administered a second daily dose of about 10 mg of lomitapide, or a pharmaceutically acceptable salt thereof, for a second dosing period;
d) measuring the fasting triglyceride levels of the patient after the second dosing period, wherein if the patient's fasting triglyceride levels are ≤1000 mg/dL while adhering to a low-fat diet, the patient is maintained at a daily dose of lomitapide, or a pharmaceutically acceptable salt thereof, of about 10 mg;
e) if the patient's measured fasting triglyceride levels are >1000 mg/dL while adhering to a low-fat diet after the second dosing period, the patient is orally administered a third daily dose of about 15-20 mg of lomitapide, or a pharmaceutically acceptable salt thereof, for a third dosing period;
f) measuring the fasting triglyceride levels of the patient after the third dosing period, wherein if the patient's fasting triglyceride levels are ≤1000 mg/dL while adhering to a low-fat diet, the patient is maintained at a daily dose of lomitapide, or a pharmaceutically acceptable salt thereof, of about 15-20 mg; and
g) if the patient's measured fasting triglyceride levels are >1000 mg/dL while adhering to a low-fat diet after the third dosing period, the patient is orally administered a fourth daily dose of about 30-40 mg of lomitapide, or a pharmaceutically acceptable salt thereof, for a fourth dosing period.

In some embodiments, methods of the present disclosure are used to treat familial chylomicronemia syndrome (FCS) in a patient in need thereof, the method comprising:
a) orally administering a first daily dose of about 5 mg of lomitapide, or a pharmaceutically acceptable salt thereof, to the patient for a first dosing period;
b) measuring the fasting triglyceride levels of the patient after the first dosing period, wherein if the patient's fasting triglyceride levels are ≤1000 mg/dL while adhering to a low-fat diet, the patient is maintained at a daily dose of lomitapide, or a pharmaceutically acceptable salt thereof, of about 5 mg;
c) if the patient's measured fasting triglyceride levels are >1000 mg/dL while adhering to a low-fat diet after the first dosing period, the patient is orally administered a second daily dose of about 10 mg of lomitapide, or a pharmaceutically acceptable salt thereof, for a second dosing period;
d) measuring the fasting triglyceride levels of the patient after the second dosing period, wherein if the patient's fasting triglyceride levels are ≤1000 mg/dL while adhering to a low-fat diet, the patient is maintained at a daily dose of lomitapide, or a pharmaceutically acceptable salt thereof, of about 10 mg;
e) if the patient's measured fasting triglyceride levels are >1000 mg/dL while adhering to a low-fat diet after the second dosing period, the patient is orally administered a third daily dose of about 15-20 mg of lomitapide, or a pharmaceutically acceptable salt thereof, for a third dosing period;
f) measuring the fasting triglyceride levels of the patient after the third dosing period, wherein if the patient's fasting triglyceride levels are ≤1000 mg/dL while adhering to a low-fat diet, the patient is maintained at a daily dose of lomitapide, or a pharmaceutically acceptable salt thereof, of about 15-20 mg;
g) if the patient's measured fasting triglyceride levels are >1000 mg/dL while adhering to a low-fat diet after the third dosing period, the patient is orally administered a fourth daily dose of about 30-40 mg of lomitapide, or a pharmaceutically acceptable salt thereof, for a fourth dosing period; and
h) measuring the fasting triglyceride levels of the patient after the fourth dosing period, wherein if the patient's fasting triglyceride levels are ≤1000 mg/dL while adhering to a low-fat diet, the patient is maintained at a daily dose of lomitapide, or a pharmaceutically acceptable salt thereof, of about 30-40 mg; and
i) if the patient's measured fasting triglyceride levels are >1000 mg/dL while adhering to a low-fat diet after the fourth dosing period, the patient is orally administered a fifth daily dose of about 40-60 mg of lomitapide, or a pharmaceutically acceptable salt thereof, for a fifth dosing period.

In embodiments, the present disclosure provides methods of treating familial chylomicronemia syndrome (FCS) in a patient in need thereof, the method comprising:
a) orally administering a first daily dose of about 5 mg of lomitapide, or a pharmaceutically acceptable salt thereof, to the patient for a first dosing period;
b) measuring the fasting triglyceride levels of the patient after the first dosing period, wherein if the patient's fasting triglyceride levels are ≤750 mg/dL while adhering to a low-fat diet, the patient is maintained at a daily dose of lomitapide, or a pharmaceutically acceptable salt thereof, of about 5 mg;
c) if the patient's measured fasting triglyceride levels are >750 mg/dL while adhering to a low-fat diet after the first dosing period, the patient is orally administered a second daily dose of about 10 mg of lomitapide, or a pharmaceutically acceptable salt thereof, for a second dosing period.

In some embodiments, the present disclosure provides methods of treating familial chylomicronemia syndrome (FCS) in a patient in need thereof, the method comprising:
a) orally administering a first daily dose of about 5 mg of lomitapide, or a pharmaceutically acceptable salt thereof, to the patient for a first dosing period;
b) measuring the fasting triglyceride levels of the patient after the first dosing period, wherein if the patient's fasting triglyceride levels are ≤750 mg/dL while adhering to a low-fat diet, the patient is maintained at a daily dose of lomitapide, or a pharmaceutically acceptable salt thereof, of about 5 mg;
c) if the patient's measured fasting triglyceride levels are >750 mg/dL while adhering to a low-fat diet after the first dosing period, the patient is orally administered a second daily dose of about 10 mg of lomitapide, or a pharmaceutically acceptable salt thereof, for a second dosing period;
d) measuring the fasting triglyceride levels of the patient after the second dosing period, wherein if the patient's fasting triglyceride levels are ≤750 mg/dL while adhering to a low-fat diet, the patient is maintained at a daily dose of lomitapide, or a pharmaceutically acceptable salt thereof, of about 10 mg;
e) if the patient's measured fasting triglyceride levels are >750 mg/dL while adhering to a low-fat diet after the second dosing period, the patient is orally administered a third daily dose of about 15 mg to about 20 mg of lomitapide, or a pharmaceutically acceptable salt thereof, for a third dosing period.

In some embodiments, methods of the present disclosure are used to treat familial chylomicronemia syndrome (FCS) in a patient in need thereof, the method comprising:
a) orally administering a first daily dose of about 5 mg of lomitapide, or a pharmaceutically acceptable salt thereof, to the patient for a first dosing period;
b) measuring the fasting triglyceride levels of the patient after the first dosing period, wherein if the patient's fasting triglyceride levels are ≤750 mg/dL while adhering to a low-fat diet, the patient is maintained at a daily dose of lomitapide, or a pharmaceutically acceptable salt thereof, of about 5 mg;
c) if the patient's measured fasting triglyceride levels are >750 mg/dL while adhering to a low-fat diet after the first dosing period, the patient is orally administered a second daily dose of about 10 mg of lomitapide, or a pharmaceutically acceptable salt thereof, for a second dosing period;
d) measuring the fasting triglyceride levels of the patient after the second dosing period, wherein if the patient's fasting triglyceride levels are ≤750 mg/dL while adhering to a low-fat diet, the patient is maintained at a daily dose of lomitapide, or a pharmaceutically acceptable salt thereof, of about 10 mg;
e) if the patient's measured fasting triglyceride levels are >750 mg/dL while adhering to a low-fat diet after the second dosing period, the patient is orally administered a third daily dose of about 20 mg of lomitapide, or a pharmaceutically acceptable salt thereof, for a third dosing period.

In some embodiments, methods of the present disclosure are used to treat familial chylomicronemia syndrome (FCS) in a patient in need thereof, the method comprising:
a) orally administering a first daily dose of about 5 mg of lomitapide, or a pharmaceutically acceptable salt thereof, to the patient for a first dosing period;
b) measuring the fasting triglyceride levels of the patient after the first dosing period, wherein if the patient's fasting triglyceride levels are ≤750 mg/dL while adhering to a low-fat diet, the patient is maintained at a daily dose of lomitapide, or a pharmaceutically acceptable salt thereof, of about 5 mg;
c) if the patient's measured fasting triglyceride levels are >750 mg/dL while adhering to a low-fat diet after the first dosing period, the patient is orally administered a second daily dose of about 10 mg of lomitapide, or a pharmaceutically acceptable salt thereof, for a second dosing period;
d) measuring the fasting triglyceride levels of the patient after the second dosing period, wherein if the patient's fasting triglyceride levels are ≤750 mg/dL while adhering to a low-fat diet, the patient is maintained at a daily dose of lomitapide, or a pharmaceutically acceptable salt thereof, of about 10 mg;
e) if the patient's measured fasting triglyceride levels are >750 mg/dL while adhering to a low-fat diet after the second dosing period, the patient is orally administered a third daily dose of about 15-20 mg of lomitapide, or a pharmaceutically acceptable salt thereof, for a third dosing period;
f) measuring the fasting triglyceride levels of the patient after the third dosing period, wherein if the patient's fasting triglyceride levels are ≤750 mg/dL while adhering to a low-fat diet, the patient is maintained at a daily dose of lomitapide, or a pharmaceutically acceptable salt thereof, of about 15-20 mg; and
g) if the patient's measured fasting triglyceride levels are >750 mg/dL while adhering to a low-fat diet after the third dosing period, the patient is orally administered a fourth daily dose of about 30-40 mg of lomitapide, or a pharmaceutically acceptable salt thereof, for a fourth dosing period.

In some embodiments, methods of the present disclosure are used to treat familial chylomicronemia syndrome (FCS) in a patient in need thereof, the method comprising:
a) orally administering a first daily dose of about 5 mg of lomitapide, or a pharmaceutically acceptable salt thereof, to the patient for a first dosing period;
b) measuring the fasting triglyceride levels of the patient after the first dosing period, wherein if the patient's fasting triglyceride levels are ≤750 mg/dL while adhering to a low-fat diet, the patient is maintained at a daily dose of lomitapide, or a pharmaceutically acceptable salt thereof, of about 5 mg;
c) if the patient's measured fasting triglyceride levels are >750 mg/dL while adhering to a low-fat diet after the first dosing period, the patient is orally administered a second daily dose of about 10 mg of lomitapide, or a pharmaceutically acceptable salt thereof, for a second dosing period;
d) measuring the fasting triglyceride levels of the patient after the second dosing period, wherein if the patient's fasting triglyceride levels are ≤750 mg/dL while adhering to a low-fat diet, the patient is maintained at a daily dose of lomitapide, or a pharmaceutically acceptable salt thereof, of about 10 mg;
e) if the patient's measured fasting triglyceride levels are >750 mg/dL while adhering to a low-fat diet after the second dosing period, the patient is orally administered a third daily dose of about 15-20 mg of lomitapide, or a pharmaceutically acceptable salt thereof, for a third dosing period;
f) measuring the fasting triglyceride levels of the patient after the third dosing period, wherein if the patient's fasting triglyceride levels are ≤750 mg/dL while adhering to a low-fat diet, the patient is maintained at a daily dose of lomitapide, or a pharmaceutically acceptable salt thereof, of about 15-20 mg;
g) if the patient's measured fasting triglyceride levels are >750 mg/dL while adhering to a low-fat diet after the third dosing period, the patient is orally administered a fourth daily dose of about 30-40 mg of lomitapide, or a pharmaceutically acceptable salt thereof, for a fourth dosing period; and
h) measuring the fasting triglyceride levels of the patient after the fourth dosing period, wherein if the patient's fasting triglyceride levels are ≤750 mg/dL while adhering to a low-fat diet, the patient is maintained at a daily dose of lomitapide, or a pharmaceutically acceptable salt thereof, of about 30-40 mg; and
i) if the patient's measured fasting triglyceride levels are >750 mg/dL while adhering to a low-fat diet after the fourth dosing period, the patient is orally administered a fifth daily dose of about 40-60 mg of lomitapide, or a pharmaceutically acceptable salt thereof, for a fifth dosing period.

In some embodiments, the first dosing period is at least two weeks. In some embodiments, the first dosing period is about two weeks.

In some embodiments, the second dosing period is at least four weeks. In some embodiments, the second dosing period is about four weeks.

In some embodiments, the third dosing period is at least about four weeks. In some embodiments, the third dosing period is about four weeks.

In some embodiments, the fourth dosing period is at least about four weeks. In some embodiments, the fourth dosing period is about four weeks.

In some embodiments, the fifth dosing period is at least about four weeks. In some embodiments, the fifth dosing period is about four weeks.

In some embodiments, the methods of the present disclosure comprise adjusting the patient's daily dose of lomitapide or a pharmaceutically acceptable salt thereof to provide fasting triglyceride levels ≤1000 mg/dL and ALT/AST levels ≤3 times the ULN. In some embodiments of the methods of the present disclosure, the patient's daily dose of lomitapide or a pharmaceutically acceptable salt thereof is increased or decreased from every 2-4 weeks to provide fasting triglyceride levels ≤1000 mg/dL and ALT/AST levels ≤3 times the ULN.

In some embodiments, the methods of the present disclosure comprise adjusting (e.g., increasing or decreasing) the patient's daily dose of lomitapide or a pharmaceutically acceptable salt thereof to provide fasting triglyceride levels to ≤about 750 mg/dL. In some embodiments of the methods of treating FCS disclosed herein, the patient's daily dose of lomitapide or a pharmaceutically acceptable salt thereof is increased or decreased by about 5-20 mg (e.g., 5 mg, 10 mg, 15 mg or 20 mg) to provide fasting triglyceride levels ≤750 mg/dL and ALT/AST levels ≤3 times the ULN.

In some embodiments, the methods of the present disclosure comprise adjusting (e.g., increasing or decreasing) the patient's daily dose of lomitapide or a pharmaceutically acceptable salt thereof to provide fasting triglyceride levels to ≤about 500 mg/dL. In some embodiments of the methods of treating FCS disclosed herein, the patient's daily dose of lomitapide or a pharmaceutically acceptable salt thereof is increased or decreased by about 5-20 mg (e.g., 5 mg, 10 mg, 15 mg or 20 mg) to provide fasting triglyceride levels ≤500 mg/dL and ALT/AST levels ≤3 times the ULN.

In some embodiments of the methods of the present disclosure, if the patient's liver aminotransferase (ALT/AST) levels are ≥5 times the upper limit of normal (ULN) after the first dosing period, the second dosing period or the third dosing period, the patient is withdrawn from lomitapide treatment. In some embodiments, the patient is withdrawn from treatment until ALT/AST levels are <3 times the ULN. In some embodiments, the method further comprises determining the patient's alkaline phosphatase, total bilirubin and INR. In some embodiments, the method further comprises reducing the patient's dose to the last dose that provided patient ALT/AST levels of <3 times the ULN. In some embodiments, the dose of lomitapide or a pharmaceutically acceptable salt thereof is reduced by no more than 50% of the immediately preceding dose level, or no more than 33% of the immediately preceding dose level, or no more than 20% of the immediately preceding dose level. In some embodiments, the dose of lomitapide or a pharmaceutically acceptable salt thereof is reduced from about 10 mg to about 5 mg. In some embodiments, the dose of lomitapide or a pharmaceutically acceptable salt thereof is reduced from about 20 mg to about 10 mg. In some embodiments, the dose of lomitapide or a pharmaceutically acceptable salt thereof is reduced from about 20 mg to about 5 mg.

In some embodiments of the methods of the present disclosure, if the patient's ALT/AST levels are from 3-5 times the ULN, the method comprises confirming the patient's ALT/AST levels are 3-5 times the ULN within one week of the elevated ALT/AST test result. In some embodiments of the methods of the present disclosure, if the elevated ALT/AST test result is confirmed, the method further comprises determining the patient's alkaline phosphatase, total bilirubin and INR. In some embodiments, the method further comprises weekly testing the patient's ALT/AST levels, alkaline phosphatase, total bilirubin and INR. In some embodiments, if the patient's total bilirubin and INR increase, ALT/AST levels increase to >5 times ULN or the patient's ALT/AST levels do not fall below <3 times ULN within about 4 weeks, the patient is withdrawn from lomitapide treatment, and in some embodiments, the method further comprises reducing the patient's dose to the last dose that provided patient ALT/AST levels of <3 times the ULN. In some embodiments, the dose of lomitapide or a pharmaceutically acceptable salt thereof is reduced by no more than 50% of the immediately preceding dose level, or no more than 33% of the immediately preceding dose level, or no more than 20% of the immediately preceding dose level. In some embodiments, the dose of lomitapide or a pharmaceutically acceptable salt thereof is reduced from about 10 mg to about 5 mg. In some embodiments, the dose of lomitapide or a pharmaceutically acceptable salt thereof is reduced from about 20 mg to about 10 mg. In some embodiments, the dose of lomitapide or a pharmaceutically acceptable salt thereof is reduced from about 20 mg to about 5 mg.

In some embodiments, the daily dose that provides fasting triglyceride levels ≤1000 mg/dL and ALT/AST levels ≤3 times the ULN is a dose of about 5 mg to about 60 mg of lomitapide or a pharmaceutically acceptable salt thereof, including about 5 mg, about 10 mg, about 15 mg, about 20 mg, about 25 mg, about 30 mg, about 35 mg, about 40 mg, about 45 mg, about 50 mg, about 55 mg or about 60 mg. In some embodiments, the daily dose that provides fasting triglyceride levels ≤1000 mg/dL and ALT/AST levels ≤3 times the ULN is selected from the group consisting of 5 mg, 10 mg, 20 mg, 30 mg, and 60 mg. In some embodiments, the daily dose is 5 mg, 10 mg, 20 mg, 30 mg, or 60 mg. In some embodiments, the daily dose is 20 mg.

In some embodiments, a daily dose of about 5 mg of lomitapide is orally administered to the patient for about 2 weeks, and then the daily dose is titrated to a dose that provides fasting triglyceride levels ≤1000 mg/dL and ALT/AST levels ≤3 times the ULN.

In some embodiments, the titration comprises administration of lomitapide, or a pharmaceutically acceptable salt thereof according to the following schedule from 5 mg/day to 10 mg/day, 20 mg/day, 40 mg/day and 60 mg/day or until an individually determined maximum dose was reached based on lipid profile (e.g., TG levels), safety (e.g., ALT/AST levels) and tolerability (e.g., persistent gastrointestinal side effects):

| | Duration Of Administration |
|---|---|
| | Before Considering Increase |
| Dosage | To Next Dosage |
| 5 mg daily | At least 2 weeks |
| 10 mg daily | At least 4 weeks |
| 20 mg daily | At least 4 weeks |
| 40 mg daily | At least 4 weeks |
| 60 mg daily | Maximum recommended dosage |

In some embodiments, the titration comprises administration of lomitapide, or a pharmaceutically acceptable salt thereof according to the following schedule from 5 mg/day to 10 mg/day, 15 mg/day, 20 mg/day, 40 mg/day and 60 mg/day or until an individually determined maximum dose was reached based on lipid profile (e.g., TG levels), safety (e.g., ALT/AST levels) and tolerability (e.g., persistent gastrointestinal side effects):

| | Duration Of Administration |
|---|---|
| | Before Considering Increase |
| Dosage | To Next Dosage |
| 5 mg daily | At least 2 weeks |
| 10 mg daily | At least 4 weeks |
| 15 mg daily | At least 4 weeks |
| 20 mg daily | At least 4 weeks |
| 40 mg daily | At least 4 weeks |
| 60 mg daily | Maximum recommended dosage |

In some embodiments, the titration comprises administration of lomitapide, or a pharmaceutically acceptable salt thereof according to the following schedule from 5 mg/day to 10 mg/day, 20 mg/day, 30 mg/day, 50 mg/day and 60 mg/day or until an individually determined maximum dose was reached based on lipid profile (e.g., TG levels), safety (e.g., ALT/AST levels) and tolerability (e.g., persistent gastrointestinal side effects):

| | Duration Of Administration |
|---|---|
| | Before Considering Increase |
| Dosage | To Next Dosage |
| 5 mg daily | At least 2 weeks |
| 10 mg daily | At least 4 weeks |
| 20 mg daily | At least 4 weeks |
| 30 mg daily | At least 4 weeks |
| 50 mg daily | At least 4 weeks |
| 60 mg daily | Maximum recommended dosage |

In some embodiments, the titration comprises administration of lomitapide, or a pharmaceutically acceptable salt thereof according to the following schedule from 5 mg/day to 10 mg/day, 20 mg/day, 40 mg/day, 50 mg/day and 60 mg/day or until an individually determined maximum dose was reached based on lipid profile (e.g., TG levels), safety (e.g., ALT/AST levels) and tolerability (e.g., persistent gastrointestinal side effects):

| | Duration Of Administration |
|---|---|
| | Before Considering Increase |
| Dosage | To Next Dosage |
| 5 mg daily | At least 2 weeks |
| 10 mg daily | At least 4 weeks |
| 20 mg daily | At least 4 weeks |
| 40 mg daily | At least 4 weeks |
| 50 mg daily | At least 4 weeks |
| 60 mg daily | Maximum recommended dosage |

### Pediatric Patients

In some embodiments, methods of the present disclosure are used to treat familial chylomicronemia syndrome (FCS) in a pediatric patient in need thereof, the method comprising:
a) orally administering a first daily dose of about 2 mg to about 5 mg of lomitapide, or a pharmaceutically acceptable salt thereof, to the patient for a first dosing period;
b) measuring the fasting triglyceride levels of the patient after the first dosing period, wherein if the patient's fasting triglyceride levels are ≤1000 mg/dL, the patient is maintained at a daily dose of lomitapide, or a pharmaceutically acceptable salt thereof, of about 2 mg to about 5 mg;
c) if the patient's measured fasting triglyceride levels are >1000 mg/dL after the first dosing period, the patient is orally administered a second daily dose of about 5 mg to about 10 mg of lomitapide, or a pharmaceutically acceptable salt thereof, for a second dosing period;
d) measuring the fasting triglyceride levels of the patient after the second dosing period, wherein if the patient's fasting triglyceride levels are ≤1000 mg/dL, the patient is maintained at a daily dose of lomitapide, or a pharmaceutically acceptable salt thereof, of about 5 mg to about 10 mg;
e) if the patient's measured fasting triglyceride levels are >1000 mg/dL after the second dosing period, the patient is orally administered a third daily dose of about 10 mg to about 20 mg of lomitapide, or a pharmaceutically acceptable salt thereof, for a third dosing period.

In some embodiments, methods of the present disclosure are used to treat familial chylomicronemia syndrome (FCS) in a pediatric patient in need thereof, the method comprising:
a) orally administering a first daily dose of about 2 mg to about 5 mg of lomitapide, or a pharmaceutically acceptable salt thereof, to the patient for a first dosing period;
b) measuring the fasting triglyceride levels of the patient after the first dosing period, wherein if the patient's fasting triglyceride levels are ≤750 mg/dL, the patient is maintained at a daily dose of lomitapide, or a pharmaceutically acceptable salt thereof, of about 2 mg to about 5 mg;
c) if the patient's measured fasting triglyceride levels are >750 mg/dL after the first dosing period, the patient is orally administered a second daily dose of about 5 mg to about 10 mg of lomitapide, or a pharmaceutically acceptable salt thereof, for a second dosing period;
d) measuring the fasting triglyceride levels of the patient after the second dosing period, wherein if the patient's fasting triglyceride levels are ≤750 mg/dL, the patient is maintained at a daily dose of lomitapide, or a pharmaceutically acceptable salt thereof, of about 5 mg to about 10 mg;
e) if the patient's measured fasting triglyceride levels are >750 mg/dL after the second dosing period, the patient is orally administered a third daily dose of about 10 mg to about 20 mg of lomitapide, or a pharmaceutically acceptable salt thereof, for a third dosing period.

In some embodiments, a daily dose of about 2 mg to about 5 mg of lomitapide is orally administered to the patient for about 2 weeks, and then the daily dose is titrated to a dose that provides fasting triglyceride levels ≤1000 mg/dL and ALT/AST levels ≤3 times the ULN.

In some embodiments, the patient's daily dose of lomitapide or a pharmaceutically acceptable salt thereof is increased or decreased from every 2-8 weeks to provide fasting triglyceride levels ≤1000 mg/dL and ALT/AST levels ≤3 times the ULN.

In some embodiments, a daily dose of about 2 mg to about 5 mg of lomitapide is orally administered to the patient for about 2 weeks, and then the daily dose is titrated to a dose that provides fasting triglyceride levels ≤750 mg/dL and ALT/AST levels ≤3 times the ULN.

In some embodiments, the patient's daily dose of lomitapide or a pharmaceutically acceptable salt thereof is increased or decreased from every 2-8 weeks to provide fasting triglyceride levels ≤750 mg/dL and ALT/AST levels <3 times the ULN.

In some embodiments the patient's age is from 5 to 10 years, from 11 to 15 years, or from 16 to 17 years.

In some embodiments, the pediatric patient in need of FCS treatment is administered a daily dose of 2 mg of lomitapide or a pharmaceutically acceptable salt thereof in the first dosing period, a daily dose of 5 mg of lomitapide or a pharmaceutically acceptable salt thereof in the second dosing period, and a daily dose of 10 mg of lomitapide or a pharmaceutically acceptable salt thereof in the third dosing period.

In some embodiments, the pediatric patient in need of FCS treatment is administered a daily dose of about 5 mg of lomitapide, or a pharmaceutically acceptable salt thereof in the first dosing period, a daily dose of about 10 mg of lomitapide, or a pharmaceutically acceptable salt thereof in the second dosing period, and a daily dose of about 20 mg of lomitapide, or a pharmaceutically acceptable salt thereof in the third dosing period.

In some embodiments, the dosing period (e.g., first dosing period, second dosing period, or third dosing period) is about or at least about 1-8 weeks, including about or at least about 1 week, about or at least about 2 weeks, about or at least about 3 weeks, about or at least about 4 weeks, about or at least about 5 weeks, about or at least about 6 weeks, about or at least about 7 weeks, or about or at least about 8 weeks, including all subranges and values therebetween.

In some embodiments, the patient's age is from 5 to 10 years and the daily dose of lomitapide in the first dosing period is 2 mg, the daily dose of lomitapide in the second dosing period is 5 mg, and the daily dose of lomitapide in the third dosing period is 10 mg. In some embodiments, the first dosing period is about or at least about 8 weeks, the second dosing period is about or at least about 4 weeks, and the third dosing period is about or at least about 4 weeks.

In some embodiments, the patient's age is from 11 to 15 years and the daily dose of lomitapide in the first dosing period is 2 mg, the daily dose of lomitapide in the second dosing period is 5 mg, and the daily dose of lomitapide in the third dosing period is 10 mg. In some embodiments, the first dosing period is about or at least about 4 weeks, the second dosing period is about or at least about 4 weeks, and the third dosing period is about or at least about 4 weeks.

In some embodiments, the patient's age is 16 to 17 years and the daily dose of lomitapide in the first dosing period is about 5 mg, the daily dose of lomitapide in the second dosing period is about 10 mg, and the daily dose of lomitapide in the third dosing period is about 20 mg. In some embodiments, the first dosing period is about or at least about 4 weeks, the second dosing period is about or at least about 4 weeks, and the third dosing period is about or at least about 4 weeks.

### Kits

In some embodiments, the present disclosure provides kits for use in treating familial chylomicronemia syndrome (FCS) in a patient in need thereof. Such kits comprise lomitapide or a pharmaceutically salt thereof. The kits of the present disclosure may be used for administering lomitapide or a pharmaceutically acceptable salt thereof at different dosage intervals, or for titrating the dose of lomitapide or a pharmaceutically acceptable salt thereof according to methods described herein. For example, the present disclosure provides kits for treating FCS in a subject, comprising at least three sets of pharmaceutical dosage units; and instructions for use.

In some embodiments, the kits of the present disclosure may comprise directions for administration. For example, the kit can include instructions to administer lomitapide or a pharmaceutically acceptable salt thereof in a suitable manner to perform the methods described herein, e.g., in a suitable dose, dosage form, dosing intervals (e.g., as described herein). In some embodiments, the informational material can include instructions to administer the lomitapide or a pharmaceutically acceptable salt thereof to a suitable patient, e.g., an adult patient with FCS, or a pediatric patient with FCS.

The kit can include one or more containers for lomitapide or a pharmaceutically salt thereof as described herein. In some embodiments, the kit contains separate containers, dividers or compartments for the composition and informational material. For example, the composition can be contained in a bottle, vial, or syringe. In some embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a composition described herein. For example, the kit can include a plurality of syringes, ampules, or foil packets each containing a single unit dose of a composition described herein. An example of such a kit is a blister pack, as typically used for the packaging of tablets, capsules and the like. The containers of the kits can be air tight, waterproof (e.g., impermeable to changes in moisture or evaporation), and/or light-tight.

The following non-limiting examples illustrate various aspects of the present disclosure.

### EXAMPLES

### Example 1

This clinical study evaluated the safety, tolerability, and efficacy of Lomitapide in patients with Familial Chylomicronemia Syndrome (FCS).

### Study Design

Patients were screened for eligibility between 12 to 6 weeks before the first dose of Lomitapide (screening period). After screening period, all enrolled patients entered a run-in phase of at least 6 weeks during which lipid-lowering therapy, daily dietary supplementation of vitamin E, essential fatty acids and a low-fat diet are instituted and stabilized. At the end of the run-in phase, the patients entered into a 26-week efficacy phase, during which they receive lomitapide in addition to their lipid-lowering therapy. Patients must follow a diet comprising <10% energy from fat per day during all the study.

### Participants

### Inclusion Criteria

Patients enrolled in the study were required to meet the following criteria to be eligible for inclusion in the study:
Aged 18 years and older at time of informed consent
History of chylomicronemia as evidenced by documentation of lactescent serum (a creamy top layer after ultracentrifugation of a fasting blood sample) or documentation of fasting TG measurement ≥885 mg/dl (10 mmol/L)
A diagnosis of Familial Chylomicronemia Syndrome by documentation of at least one of the following:
   Confirmed homozygote, compound heterozygote or double heterozygote for loss of—function mutations in genes causing FCS (such as LPL, APOC2, APOA5, GPIHBP1, or LMF1).
Fasting TG≥750 mg/dl (8.4 mmol/L) at screening. If the fasting TG<750 mg/dl up to two additional tests may be performed in order to qualify.
History of pancreatitis (defined as documented diagnosis of acute pancreatitis or hospitalization for severe abdominal pain consistent with acute pancreatitis and for which no alternate diagnosis was made).
No requirement for liver biopsy at baseline but MRI at baseline and Week 26 and LFTs at each monthly visit
Availability of serum for liver biomarker assessment at baseline and Week 26
Willing to follow a diet comprising <10% energy from fat per day during the study
Satisfy one of the following:
   Females: non-pregnant and non-lactating; surgically sterile (e.g., tubal occlusion, hysterectomy, bilateral salpingectomy, bilateral oophorectomy), post-menopausal (defined as 12 months of spontaneous amenorrhea in females >55 years of age or, in females ≤55 years, 12 months of spontaneous amenorrhea without an alternative medical cause and FSH levels in the postmenopausal range for the laboratory involved), abstinent, or if engaged in sexual relations of child-bearing potential, patient is using an acceptable contraceptive method from time of signing the informed consent form until 13 weeks after the last dose of study drug administration.
   Males: Surgically sterile, abstinent or if engaged in sexual relations with a female of child-bearing potential, patient is utilizing an acceptable contraceptive method from the time of signing the informed consent form until 13 weeks after the last dose of study drug administration.

### Exclusion Criteria

Diabetes mellitus with any of the following:
   Newly diagnosed within 12 weeks of screening
   HbA1c≥9.0% at screening
   Recent change in anti-diabetic pharmacotherapy
   Anticipated need to change dose or type of medication during the treatment period of the study
   Current use of GLP-1 agonists
Severe hypertriglyceridemia other than due to FCS
Active pancreatitis within 4 weeks prior to screening
History within 6 months of screening of acute or unstable cardiac ischemia, stroke, transient ischemic attack or unstable congestive cardiac failure requiring a change in medication or major surgery within 3 months of screening.
Any of the following laboratory values at screening:
Hepatic:
   Total bilirubin >upper limit of normal (ULN), unless prior diagnosis and documentation of Gilbert's syndrome in which case total bilirubin must be ≤3 mg/dl
   AST>2.0×ULN
   ALT>2.0×ULN
Renal:
   Persistently positive (2 out of 3 consecutive tests ≥1+) for protein on urine dipstick. In the event of positive test eligibility may be confirmed by a quantitative total urine protein measurement of <500 mg/24 h
   Persistently positive (2 out of 3 consecutive tests ≥trace positive) for blood or urine dipstick. In the event of positive test eligibility may be confirmed with urine microscopy showing ≤5 red blood cells per high power field
   Estimated creatinine clearance calculated according to the formula of Cockcroft and Gault <50 mL/min
   Any other laboratory abnormalities which, in the opinion of the Investigator or the Sponsor, would make the patient unsuitable for inclusion
Uncontrolled hypertension (BP >160/100 mmHg).
History of bleeding diathesis or coagulopathy or clinically significant abnormality in coagulation parameters at screening.
History of heart failure with NYHA greater than Class II.
Active infection requiring systemic antiviral or antimicrobial therapy that will not be completed prior to begin treatment with Lomitapide (Baseline Visit).
Known history of or positive test for human immunodeficiency virus (HIV), hepatitis C or chronic hepatitis B.
Malignancy within 5 years, except for basal or squamous cell carcinoma of the skin or carcinoma in situ of the cervix that has been successfully treated.
Treatment with another investigational drug, biological agent, or device within one month of screening, or 5 halflives of investigational agent, whichever is longer.
Unwilling to comply with lifestyle requirements.
Known lactose intolerance
Use of the following:
   Statins, omega-3 fatty acids or fibrates unless on a stable dose for at least 3 months prior to screening and dose and regimen expected to remain constant during the treatment period. Patients taking omega-3 fatty acids should make every effort to remain on the same brand throughout the study
   Nicotinic acid or derivatives of nicotinic acid within 4 weeks prior to screening
   Systemic corticosteroids or anabolic steroids within 6 weeks prior to screening unless approved by the Investigator
   Atypical antipsychotic medications unless on a stable dose for at least 4 weeks prior to screening and dose and regimen expected to remain constant during the treatment period
   Glybera gene therapy within 2 years prior to screening
   Oral anticoagulants (e.g., warfarin, dabigatran, rivaroxaban, and apixaban) unless on a stable dose for at least 4 weeks prior to screening and regular clinical monitoring is performed
   Tamoxifen, estrogens or progestins unless on a stable dose for at least 4 months prior to screening and dose and regimen expected to remain constant during the treatment period;
   Plasma apheresis within 4 weeks prior to screening or planned during the study
   Any of the medication unless stable at least 4 weeks prior to screening;
Blood donation of 50 to 499 mL within 30 days of screening or of >499 mL within 60 days of screening
Have any other conditions, which, in the opinion of the Investigators or the Sponsor would make the patient unsuitable for inclusion, or could interfere with the patient participating in or completing the study.

### Treatment

All patients who meet the eligibility criteria, and after 6-week diet period (run-in) were treated with Lomitapide 5 mg, 10 mg, 15 mg, 20 mg, 30 mg, 40 mg, 50 mg up to 60 mg daily based on lipid profile and tolerability. Patients remain at their maximum dose until the end of 26 weeks.

Treatment was initiated at 5 mg once daily, and the dose titrated based on acceptable safety/tolerability. After a minimum of 2 weeks at 5 mg once daily the dose was then titrated at a minimum of 4-week intervals to 10 mg, 20 mg, 40 mg, and 60 mg/day (e.g., as shown in the table below) or until an individually determined maximum dose (e.g., in the range of 5-60 mg/day) was reached based on lipid profile, and safety/tolerability. Dose adjustments were also made according to liver transaminase levels. If subjects experienced confirmed alanine aminotransferase (ALT) or aspartate aminotransferase (AST) elevations between 3-5×ULN, or >100 U/L but <200 U/L above the baseline value, the dose of lomitapide was reduced to the previously tolerated dose level, with the possibility to re-escalate dose once transaminase elevations were resolved. Once a maximum dose was established, patients remained on this dose up to Week 26.

The fastest up-titration schedule permitted by the protocol is as follows:

| | Duration Of Administration |
|---|---|
| | Before Considering Increase |
| Dosage | To Next Dosage |
| 5 mg daily | At least 2 weeks |
| 10 mg daily | At least 4 weeks |
| 20 mg daily | At least 4 weeks |
| 40 mg daily | At least 4 weeks |
| 60 mg daily | Maximum recommended dosage |

Lomitapide should be administered once daily at bedtime, with a glass of water and without food.

Lipid levels and safety indices, including liver function tests, were evaluated at baseline, before each dose increase, and then every 4 weeks until week 26.

Following completion of the study at Week 26, eligible subjects completing the treatment phase were given the option to enter under an expanded access program), in which subjects continued to receive lomitapide on the basis of compassionate use.

### Endpoints

The Primary Endpoint was percent change in tryglycerides (TG) at the maximum tolerated dose compared to baseline after 26 weeks of treatment in combination with other lipid lowering therapy in patients with Familial Chylomicronemia Syndrome (FCS).

Secondary endpoints include other lipid parameters, hepatic fat and liver stiffness and Chylomicron kinetics of Lomitapide in combination with other lipid lowering agents in term of changes at 26 weeks from baseline. Including:
a) Percent change in TC, non-HDL-C, LDL-C, VLDL, Lp(a), as well as apolipoproteins B and A1.
b) Safety of Lomitapide in patients with FCS assessed by changes in laboratory parameters, electrocardiogram, physical examinations and weight.
c) Record of episodes of pancreatitis
d) Change in hepatic fat liver stiffness measured by MRI and/or Transient Elastography (Fibroscan).
e) Chylomicron kinetics.

To enable collection of data for the primary and other endpoints a fasting lipid and safety panel, including liver function tests, was obtained at baseline, prior to each dose escalation, and every 4 weeks thereafter until Week 26. Blood was drawn at baseline and at each visit following a 12 hour fast. Testing included a standard metabolic panel, a complete blood count, urinalysis. Ten patients underwent a metabolic study to determine postprandial chylomicron metabolism and fatty acid profile. Available samples were separated by centrifugation and immediately stored at −80° C. and were shipped in dry ice at the end of the study for analysis.

Lipid and lipoprotein analyses were conducted on serum samples. Total cholesterol (TC), high density lipoprotein-cholesterol (HDL-C), and TGs were measured enzymatically. Non-HDL-C was calculated by subtracting HDL-C levels from TC levels. ApoA-I, apoB and Lp(a) were measured by immunonephelometry.

Percentage hepatic fat was determined by magnetic resonance imaging (MRI) at baseline and Week 26. The MRI protocol included a dual-phase sequence and the IDEAL IQ sequence. Post-processing software, provided by the manufacturer, was used to generate fat fraction maps. A radiologist trained in abdominal imaging imaged four 1 cm² regions of interest (ROIs) to measure in-phase signal intensities of the liver parenchyma. ROIs were copied from the in-phase images to the opposed-phase to ensure identical size and location. Focal hepatic lesions, major branches of portal or hepatic veins, and artifacts were avoided. The mean of the signal intensity of the liver was calculated as the average value of the four signal intensities of the liver parenchyma both in the in-phase and in the opposed phase. The hepatic fat fraction was then calculated with the following formula: 100×(signal intensity_{IP}−signal intensity_{OP})/(2×signal intensity_{IP}). Finally, ROIs were also copied in the HFF Axial IDEAL IQ map to ensure identical size and location (this map was not available in one patient). The liver ROIs placed on the IDEAL-IQ fat fraction reconstruction were used to generate estimates of percentage fat.

Noninvasive quantification of liver stiffness (LSM) in kPa (estimated fibrosis score: F0 to F1: 2-7 kPa; F2: 7.5-10 kPa; F3: 10-14 kPa; F4: >10 kPa) was measured by ultrasound-based transient elastography using FibroScan® or shear wave elastography. Non-alcoholic fatty liver disease fibrosis score (NFS) and fibrosis-4 (FIB-4) score were calculated according to Angulo et al (2007) (Angulo P, Hui J M, Marchesini G, et al. The NAFLD fibrosis score: a noninvasive system that identifies liver fibrosis in patients with NAFLD. Hepatology 2007; 45(4), which is incorporated by reference herein.

Adverse events (AEs) were coded using MedDRA, Version 11.0. AEs were judged by the investigators as not related, unlikely, possibly, probably or definitely related to study drug and were reviewed regularly by an independent Data and Safety Monitoring Board.

### Statistical Analysis

Numeric parameters were expressed as median values and 97.5% confidence intervals, while dichotomous variables were expressed as proportions. Differences in numeric parameters were evaluated by the Exact Wilcoxon-Mann-Whitney Test (R CRAN “coin” package). Differences in proportions were evaluated by the Chi Square test. Percentual reductions of numeric variables at Week 26 were expressed as median values (with 97.5% confidence intervals) of the individual patients' variations from Week 0. Correlation of TG percent reduction with lomitapide dose was calculated by partial Spearman's correlation adjusting for TG baseline absolute values (R CRAN ‘ppcor’ package). All calculations were performed by the R statistical software Version 4.04 under the RStudio Version 1.3.1093 interface.

### Results

Eighteen adult patients with FCS were recruited and enrolled in the study, with 100% completing the study (26 weeks) and 11 entered the expanded access program. Characteristics of the patients at baseline is shown in Table 1, and the genotype and baseline lipid profile of FCS patients enrolled in the study is shown in Table 2.

TABLE 1
Characteristics of the patients at baseline

| Characteristics | Value | |
|---|---|---|
| Mean age (range), years | 46.55 | (19-75) |
| Sex, M/F, n (%) | 8/10 | (44.4/55.6) |
| Female | 10 | (55.6) |
| Male | 8 | (44.4) |
| Median body mass index | 22.75 | (20.2-25.8) |
| (97.5% CI), kg/m² | | |
| Median triglycerides | 1803.5 | (1452-2391) |
| (97.5% CI), mg/dL | | |
| History of acute | 18 | (100) |
| pancreatitis, n (%) | | |
| History of recurrent | | |
| acute pancreatitis, n (%) | | |
| Baseline use of n-3 fatty | 18 | (100) |
| acids, fibrates, or both, n (%) | | |
| Genetic mutations, n (%) | | |
| LPL | 14 | (78) |
| APOC2 | 2 | (11) |
| APOA5 | 0 | |
| LMF1 | 0 | |
| GPIHBPI | 0 | |
| LPL/APOA5 | 1 | (5.5) |
| LPL/GPIHBPI | 1 | (5.5) |

To convert the values for triglycerides from mg/dL to mmol/L multiply by 0.01129
LPL, lipoprotein Lipase;
APOC2, apoprotein C2;
APOA5, apoprotein A5;
LMF1, lipase maturation factor 1;
GPIHBPI, glycosylphosphatidylinositol-anchored high-density lipoprotein-binding protein 1

TABLE 2
Genotype and baseline lipid profile of FCS patients enrolled in the study

| Subject | | | TC, | HDL-C, | TG, |
|---|---|---|---|---|---|
| ID | Gene | Mutations | mg/dL | mg/dL | mg/dL |
| 10001 | LPL | c.250-1 G > C | 172 | 14 | 1965 |
| | | (IVS2) | | | |
| 10002 | LPL | c.829 G > A | 297 | 10 | 2622 |
| | | (p.Asp277Asn) | | | |
| 10004 | APOC2 | c.177 C > A | 177 | 19 | 1012 |
| | | p.Tyr59Ter | | | |
| 10005 | LPL/APOA5 | c.1174 C > G, | 192 | 14 | 1582 |
| | | (p.Leu392Val)/ | | | |
| | | c.457G > A | | | |
| | | (p.Val153Met) | | | |
| 20001 | LPL/GPIHBPI | c.984G > T | 252 | 18 | 2135 |
| | | (p.Met328Ile)/ | | | |
| | | c.41G > T | | | |
| | | (p.Cys14Phe) | | | |
| 20002 | LPL | c.1019-2A > T | 224 | 17 | 1531 |
| | | (IVS6) | | | |
| 20003 | LPL | c.832_833delTC | 290 | 21 | 2950 |
| | | (p.Asp277Asp fsX4) | | | |
| 20004 | LPL | c.987C > A | 305 | 13 | 2391 |
| | | (p.Tyr329Ter) | | | |
| 20005 | LPL | c.651delT | 232 | 16 | 2498 |
| | | (p.Pro217Pro Fs34X) | | | |
| 20006 | LPL | c.326T > C | 192 | 12 | 2150 |
| | | (p.Ile109Thr) | | | |
| 20007 | LPL | c.644G > A | 98 | 17 | 810 |
| | | (p.Gly215Glu) | | | |
| 20008 | LPL | c.1019-2A > T | 213 | 16 | 1642 |
| | | (IVS6) | | | |
| 20009 | LPL | c.621C > G | 198 | 14 | 1593 |
| | | (p.Asp207Glu) | | | |
| 20010 | APOC2 | c.274C > T | 176 | 16 | 1452 |
| | | (p.Gln92Ter) | | | |
| 20011 | LPL | c.542G > A | 168 | 20 | 1101 |
| | | (p.Gly181Asp) | | | |
| 30001 | LPL | c.755C > T | 154 | 17 | 1358 |
| | | (p.Ile252Thr) | | | |
| | | c.(?_−1)_(*1_?)del | | | |
| 30002 | LPL | c.621C > G | 232 | 11 | 2224 |
| | | (p.Asp207Glu) | | | |
| 30003 | LPL | c.1174C > G | 465 | 15 | 4151 |
| | | (p.Leu392Val) | | | |

TC, total cholesterol;
HDL-C, high-density lipoprotein cholesterol,
TG, triglycerides;
LPL, lipoprotein lipase;
ApoC2, apolipoprotein C2;
APOA5, apolipoprotein A5;
GPIHBPI, glycosylphosphatidylinositol-anchored high-density lipoprotein-binding protein 1

All 18 subjects were either homozygotes, compound heterozygotes or double heterozygotes for mutations in genes affecting the intravascular lipolytic chylomicron cascade. All subjects were undergoing treatment with omega-3 fatty acids, fibrates, or both. Despite lipid lowering treatment, triglyceride levels were markedly elevated at baseline. Compliance with lomitapide dosing, defined as >80% of capsules taken, was 96% during the study.

As shown in Table 3, below treatment was initiated at a starting daily dose of 5 mg of Lomitapide and the dose titrated based on lipid profile and tolerability.

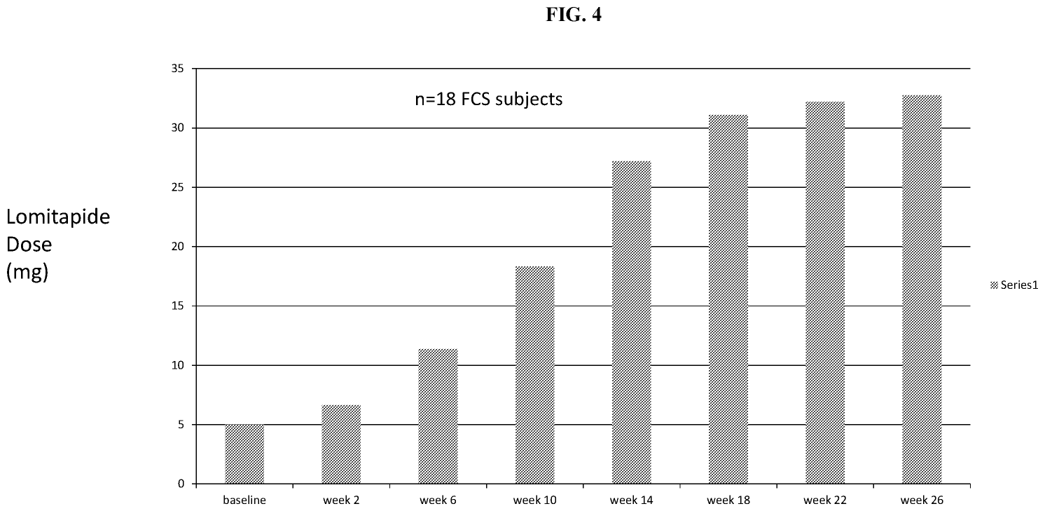

The mean Lomitapide daily dose over the course of the study is shown in FIG. 4.

TABLE 3
Lomitapide daily dose (mg) by patient

| | | Lomitapide Dose (mg) | | | | | | |
|---|---|---|---|---|---|---|---|---|
| | | Week | Week | Week | Week | Week | Week | Week |
| Patient | Baseline | 2 | 6 | 10 | 14 | 18 | 22 | 26 |
| 10001 | 5 | 10 | 20 | 20 | 20 | 20 | 20 | 20 |
| 10002 | 5 | 10 | 20 | 30 | 30 | 30 | 30 | 30 |
| 10004 | 5 | 10 | 20 | 30 | 30 | 15 | 15 | 15 |
| 10005 | 5 | 5 | 5 | 5 | 5 | 5 | 5 | 5 |
| 20001 | 5 | 5 | 10 | 20 | 40 | 50 | 60 | 60 |
| 20002 | 5 | 5 | 10 | 20 | 40 | 40 | 60 | 60 |
| 20003 | 5 | 5 | 10 | 20 | 30 | 15 | 15 | 15 |
| 20004 | 5 | 5 | 10 | 20 | 40 | 60 | 60 | 60 |
| 20005 | 5 | 5 | 10 | 20 | 40 | 60 | 50 | 50 |
| 20006 | 5 | 5 | 10 | 20 | 40 | 40 | 40 | 40 |
| 20007 | 5 | 5 | 10 | 10 | 10 | 10 | 10 | 10 |
| 20008 | 5 | 5 | 10 | 20 | 30 | 40 | 40 | 40 |
| 20009 | 5 | 5 | 10 | 20 | 30 | 20 | 20 | 20 |
| 200010 | 5 | 5 | 10 | 20 | 30 | 40 | 40 | 40 |
| 200011 | 5 | 5 | 10 | 20 | 30 | 40 | 40 | 40 |
| 30001 | 5 | 10 | 10 | 15 | 15 | 20 | 20 | 20 |
| 30002 | 5 | 10 | 20 | 30 | 40 | 40 | 40 | 40 |
| 30003 | 5 | 10 | 20 | 30 | 30 | 30 | 30 | 30 |

Among the 18 subjects who completed the study, maximum dose by Week 26 was 5 mg in one subject; 10 mg in two subjects; 15 mg in one patient; 20 mg in three patients; 30 mg in two patients; 40 mg in five patients; 50 mg in one patient and 60 mg in three patients. The median lomitapide maximum dose was 35 mg/day.

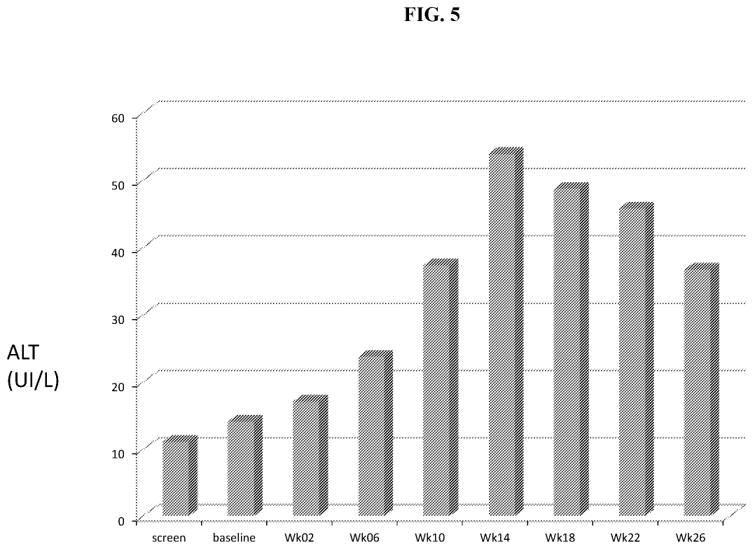

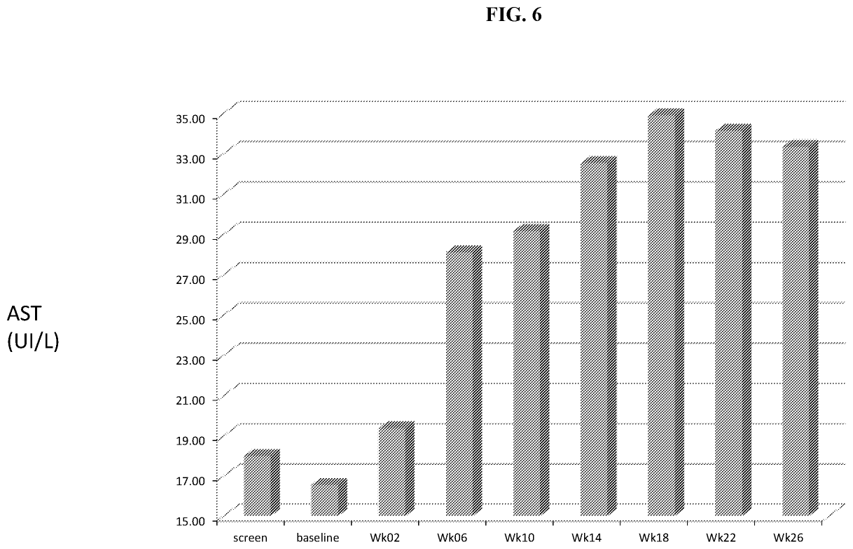

The mean alanine aminotransferase (ALT), and aspartate aminotransferase (AST), levels (ul/L) from screening to week 26 of the study are depicted in FIGS. 5 and 6.

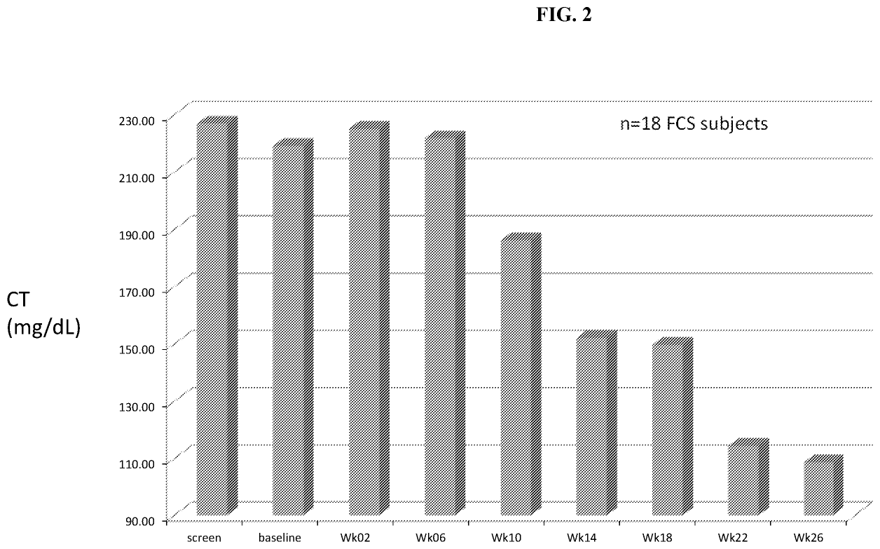

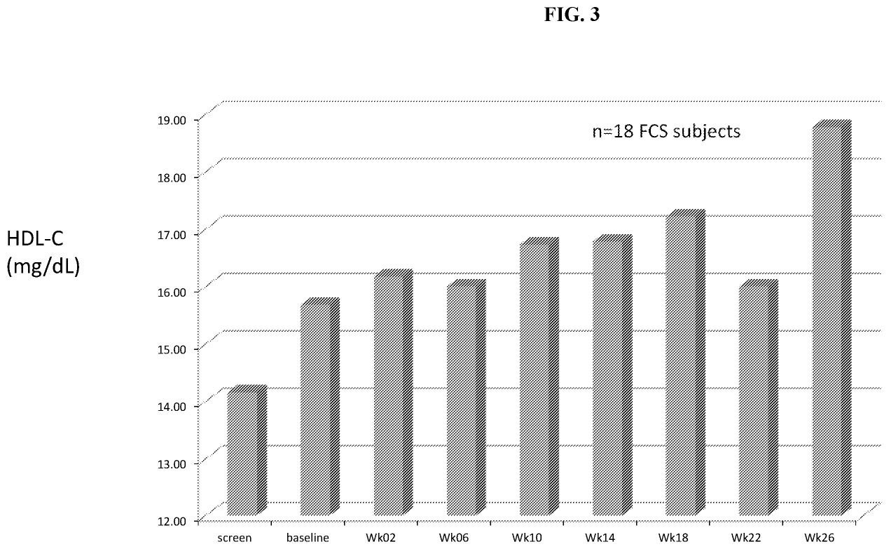

Changes in TC and HDL-C levels following treatment with lomitapide are shown in FIG. 2 and FIG. 3 respectively.

Triglyceride (TG) levels (mg/dL) in FCS patients over the 26 week treatment with lomitapide is shown in Table 4 below.

TABLE 4
Triglyceride levels (mg/dL) by patient

| | | | | | | | | | Hepatic |
|---|---|---|---|---|---|---|---|---|---|
| | | Triglyceride levels (mg/dL) | | | | | | | Fat |
| | | Week | Week | Week | Week | Week | Week | Week | Frac. (% |
| Patient | Baseline | 2 | 6 | 10 | 14 | 18 | 22 | 26 | wk. 26) |
| 10001 | 1965 | 948 | 806 | 461 | 477 | 360 | 260 | 292 | 50 |
| 10002 | 2622 | 1269 | 1117 | 1168 | 1349 | 954 | 754 | 1519 | No fatty |
| | | | | | | | | | liver |
| 10004 | 1012 | 862 | 363 | 1303 | 513 | 632 | 843 | 318 | 41 |
| 10005 | 1582 | 297 | 745 | 545 | 542 | 292 | 1341 | 231 | 20 |
| 20001 | 2135 | 1193 | 4310 | 1919 | 2403 | 303 | 1102 | 1625 | 15 |
| 20002 | 1531 | 2008 | 1181 | 1078 | 802 | 1188 | 513 | 201 | No fatty |
| | | | | | | | | | liver |
| 20003 | 2950 | 1528 | 2467 | 1884 | 364 | 251 | 196 | 273 | 34 |
| 20004 | 2391 | 2468 | 2315 | 2280 | 1503 | 1809 | 382 | 219 | Not |
| | | | | | | | | | tested |
| 20005 | 2498 | 2833 | 2267 | 2770 | 3431 | 2365 | 1820 | 705 | Not |
| | | | | | | | | | tested |
| 20006 | 2150 | 1532 | 2035 | 2214 | 1206 | 1902 | | 1117 | 5 |
| 20007 | 810 | 700 | 460 | 198 | 90 | 141 | 373 | 276 | Moderate |
| | | | | | | | | | Steatosis |
| 20008 | 1642 | 1239 | 1292 | 567 | 529 | 349 | 317 | 505 | No fatty |
| | | | | | | | | | liver |
| 20009 | 1593 | 1653 | 1473 | 1169 | 1791 | 2228 | 1619 | 801 | Mild |
| | | | | | | | | | steatosis |
| 200010 | 1452 | 1550 | 373 | 578 | 531 | 424 | 194 | 71 | No fatty |
| | | | | | | | | | liver |
| 200011 | 1101 | 1649 | 2067 | 516 | 387 | 552 | 240 | 750 | Mild |
| | | | | | | | | | steatosis |
| 30001 | 1358 | 877 | 507 | 328.5 | 222.8 | 531 | 93 | 70 | Not |
| | | | | | | | | | tested |
| 30002 | 2224 | 3101 | 3920 | 1096 | 1598 | 3317 | 1408 | 1818 | Not |
| | | | | | | | | | tested |
| 30003 | 4151 | 3604 | 2673 | 1900 | 282 | 1738 | 1502 | 196 | Not |
| | | | | | | | | | tested |

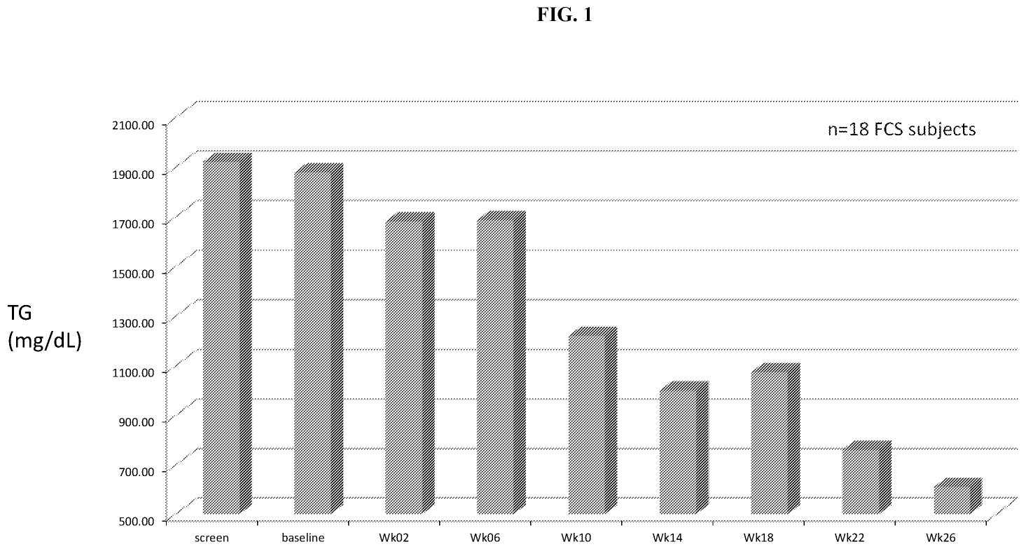

As shown in Table 4 and FIG. 1, there was a significant reduction in fasting triglyceride levels compared to baseline following treatment with Lomitapide.

When patients crossed the threshold of <1000 mg/dL triglycerides they were receiving a median dose of 10 mg of lomitapide, and mean dose of 18 mg of lomitapide.

When patients crossed the threshold of <750 mg/dL triglycerides they were receiving a median dose of 20 mg of lomitapide, and mean dose of 24 mg of lomitapide.

When patients crossed the threshold of <500 mg/dL triglycerides they were receiving a median dose of 20 mg of lomitapide, and mean dose of 27 mg of lomitapide.

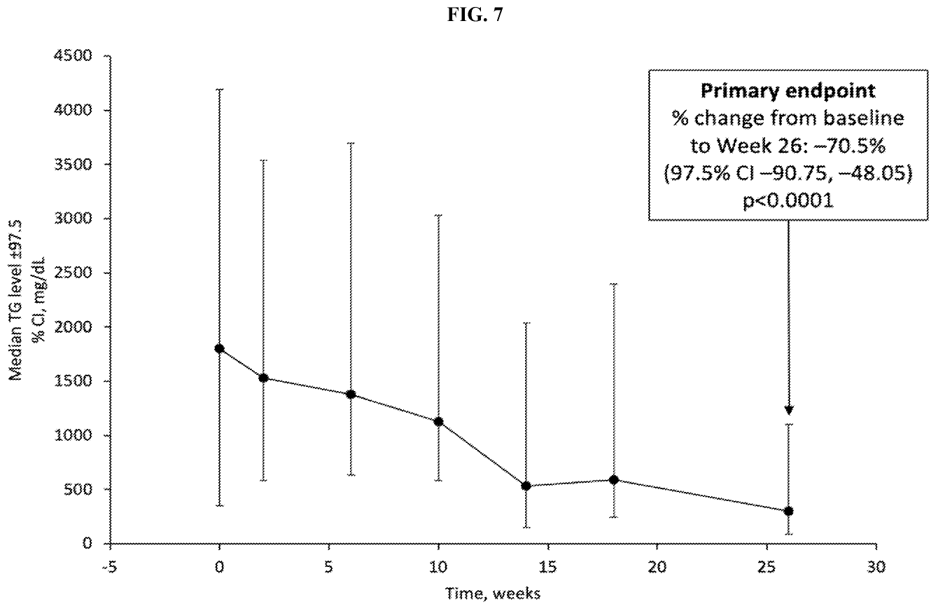

Median TG levels decreased from 1803.5 mg/dL (97.5% CI 1452-2391 mg/dL) at baseline to median fasting TG levels of 305.0 mg/dL (97.5% CI 209-801) mg/dL at the end of the study (Week 26). There was a statistically significant reduction in TG levels of 70.5% from baseline (97.5% CI, −90.7 to −48.0, p<0.0001) (Table 5, FIG. 7). Changes from baseline to Week 26 for key secondary end points (TC, HDL-C, Non-HDL-C, ApoB, ApoA-I and Lp(a)) are shown in Table 5.

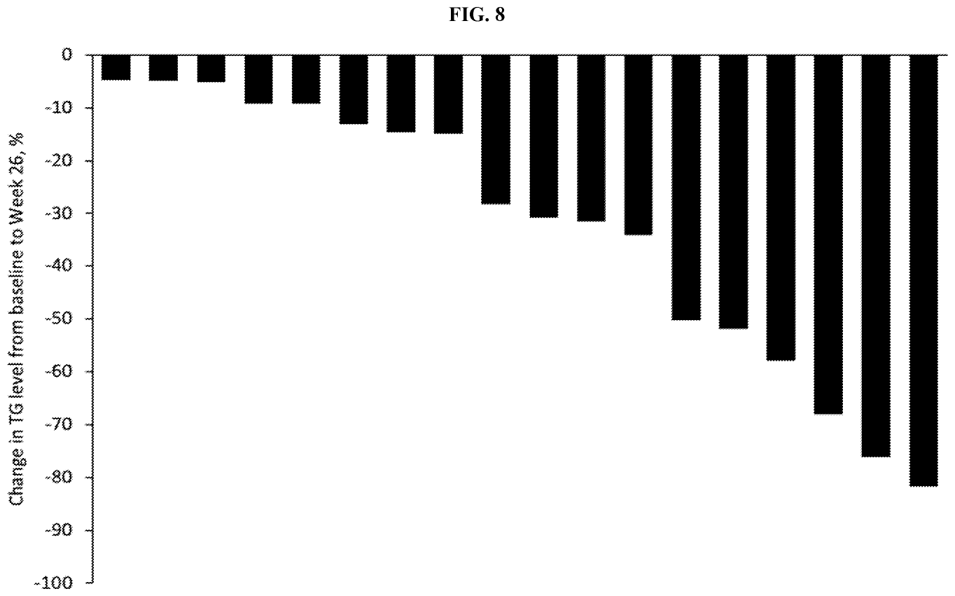

At Week 26, six subjects (33.3%) experienced decreases in TG up to 50% (18.25-49.71%). Twelve patients (66.7%) experienced TG reduction >50% and of those nine patients (50%) underwent a reduction >70%. Median triglyceride plasma levels <1000 mg/dL was achieved in 77.8% (n=14) patients. At Week 26 Thirteen subjects achieved TG levels <750 mg/dL (<8.5 mmol/L) at Week 26, with ten (55.6%) of these patients achieving TG levels <500 mg/dL (<5.6 mmol/L). FIG. 8 shows a waterfall plot of the individual percent change in TGs for all 18 subjects at Week 26. The individual percent reductions shown in FIG. 8 were not correlated with the lomitapide dose (partial correlation Spearman Rho 0.142, p-value 0.587).

No significant differences were observed for Lp(a) and hsCRP levels from baseline to Week 26 (Table 5). An increase of 20.7% in HDL cholesterol was observed (p<0.0015) at Week 26, while apoA-I levels were reduced by −28.1% (97.5% CI −31.8, −5.6, <0.0001; Table 2).

TABLE 5
Changes in lipid parameters and hsCRP from baseline to Week 26

| | | | Median of | |
|---|---|---|---|---|
| | | | individual | |
| | | | changes from | |
| | Baseline | Week 26 | baseline % | p |
| Parameter | (n = 18) | (n = 18) | (97.5% CI) | value* |

Primary endpoint

| | | | | | | | |
|---|---|---|---|---|---|---|---|
| TG, mg/dL | 1803.5 | (1452-2391) | 305.0 | (209-801) | −70.5 | (−90.7, −48.0) | <0.0001 |

Secondary endpoints

| | | | | | | | |
|---|---|---|---|---|---|---|---|
| TC, mg/dL | 205.5 | (176-252) | 94 | (69-132) | −51.7 | (−60.8, −33.7) | <0.0001 |
| HDL-C, mg/dL | 16 | (14-17) | 18 | (16-22) | +20.7 | (+33.3, 15.0) | <0.012 |
| non-HDL-C, mg/dL | 184 | (148-234) | 90.0 | (44-109) | −50.0 | (−66.5, −26.4) | <0.0001 |
| Lp(a), nmol/L | 6 | (3-11) | 5.5 | (4-30) | +40.5 | (−20, +200 | <0.507 |
| ApoB, mg/dL | 81.85 | (64.7-87.2) | 39.25 | (25.0-50.6) | −43.8 | (−66.3, −25.2) | <0.0001 |
| ApoA-I, mg/dL | 93.7 | (86.1-99.1) | 74.95 | (68.7-87.5) | −23.0 | (−31.8, −4.6) | <0.0001 |
| hsCRP, mg/L | 1.2 | (0.15-4.15) | 1.24 | (0.3-2.32) | 9.8 | (−0.7, +0.8) | <0.862 |

*Exact Wilcoxon-Mann-Whitney Test
Data expressed as median with 97.5% confidence Intervals (CI) in brackets
TG, triglycerides;
TC, total cholesterol;
HDL-C, high-density lipoprotein cholesterol;
non-HDL-C, non-HDL cholesterol;
Lp(a), lipoprotein (a);
ApoB, apolipoprotein B;
ApoA-I, apolipoprotein A-I;
hsCRP, high-sensitivity C-reactive protein

Treatment with lomitapide was well tolerated and all patients completed 26 weeks of treatment.

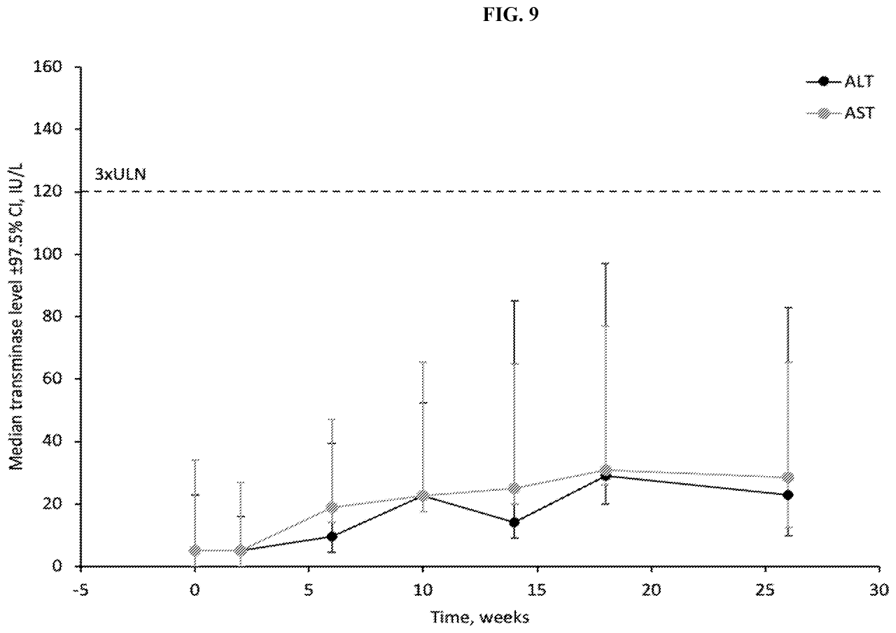

Adverse events were mild to moderate and mostly related to gastrointestinal tolerability and liver enzyme elevations. Median ALT and AST levels over time are shown in FIG. 9. No subject discontinued treatment permanently due to liver transaminase elevations and all increases were managed either by dose reduction or temporary interruption of lomitapide as per protocol. No subject experienced elevations in bilirubin or alkaline phosphatase levels.

Liver MRI imaging was used to determine the hepatic fat fraction, and revealed an increase in hepatic fat content which was between 30-50% at week 26 in 3 patients

No significant changes were seen for non-invasive liver fibrosis measurements including quantification of liver stiffness, FIB-4 and NFS scores (Table 6).

No patient experienced an episode of acute pancreatitis or severe abdominal pain during lomitapide treatment.

TABLE 6
Changes in markers of liver fibrosis from baseline to Week 26

| | | | | | Individual | | |
|---|---|---|---|---|---|---|---|
| | | | | | change from | | |
| | | | | | baseline, % | | p- |
| Parameter (N*) | Baseline | | Week 26 | | (95% CI) | | value* |
| LSM, kPa (14) | 5.7 | (5.0-6.6) | 5.5 | (4.6-7.5) | (−38.46, +5.63) | | 0.9593 |
| Hepatic fat | 12.0 | (2-30) | 32.5 | (6-50) | +146.4 | (+14.3, +900) | <0.041 |
| content on | | | | | | | |
| MRI, % (9) | | | | | | | |
| NFS (18) | 0.786 | (0.208-1.760) | 0.428 | (0.112-1.247) | −51.69 | (−0.41, −86.06) | <0.0582 |
| FIB-4 (18) | 0.76 | (0.48-1.12) | 1.03 | (0.58-1.76) | +38.44 | (−18.40, +106.00) | <0.0538 |

*Exact Wilcoxon-Mann-Whitney Test
Data expressed ad median with 97.5% confidence Intervals (CI) in brackets
LSM: Liver stiffness measurement;
kPa: kilopascals;
MRI: Magnetic resonance imaging;
NAFLD fibrosis score (NFS);
FIB-4: Fibrosis-5;
(N*): number of subjects with data available at baseline and at 26 weeks

The study met its primary efficacy endpoint of reduction in TG levels at Week 26. The existing armamentarium of fibrates and omega-3 compounds, in combination with a difficult dietary regimen, are ineffective in FCS, and other therapies e.g., antisense oligonucleotide volanesorsen has limitations in tolerability and efficacy. Therefore lomitapide has the potential to fulfil an established unmet medical need in this difficult-to-treat condition.

### EMBODIMENTS

1. A method of treating familial chylomicronemia syndrome (FCS) in a patient in need thereof, the method comprising:
   a) orally administering a first daily dose of about 5 mg of lomitapide, or a pharmaceutically acceptable salt thereof, to the patient for a first dosing period;
   b) measuring the fasting triglyceride levels of the patient after the first dosing period, wherein if the patient's fasting triglyceride levels are ≤1000 mg/dL while adhering to a low-fat diet, the patient is maintained at a daily dose of lomitapide, or a pharmaceutically acceptable salt thereof, of about 5 mg;
   c) if the patient's measured fasting triglyceride levels are >1000 mg/dL while adhering to a low-fat diet after the first dosing period, the patient is orally administered a second daily dose of about 10 mg of lomitapide, or a pharmaceutically acceptable salt thereof, for a second dosing period;
   d) measuring the fasting triglyceride levels of the patient after the second dosing period, wherein if the patient's fasting triglyceride levels are ≤1000 mg/dL while adhering to a low-fat diet, the patient is maintained at a daily dose of lomitapide, or a pharmaceutically acceptable salt thereof, of about 10 mg;
   e) if the patient's measured fasting triglyceride levels are >1000 mg/dL while adhering to a low-fat diet after the second dosing period, the patient is orally administered a third daily dose of about 20 mg of lomitapide, or a pharmaceutically acceptable salt thereof, for a third dosing period.
2. The method of embodiment 1, wherein the patient is a confirmed homozygote, compound heterozygote or double heterozygote for one or more loss-of-function mutations in genes causing FCS.
3. The method of any one of embodiments 1-2, wherein the patient has a history of pancreatitis.
4. The method of any one of embodiments 1-3, wherein the patient's post-heparin plasma lipoprotein lipase (LpL) activity is <20% of normal.
5. The method of any one of embodiments 1-4, wherein the patient has confirmed presence of LpL inactivating antibodies.
6. The method of any one of embodiments 1-5, wherein the patient's FCS is refractory to plasma LDL apheresis.
7. The method of any one of embodiments 1-6, wherein the lomitapide is administered as an adjunct to a low-fat diet and other lipid-lowering treatments (such as, statin, ezetimibe, nicotinic acid, bile acid sequestrant, fibrate or LDL apheresis).
8. The method of any one of embodiments 1-7, wherein the low-fat diet comprises a diet wherein less than 10% of patient's total calories are from fat.
9. The method of any one of embodiments 1-8, wherein the patient expresses a microsomal triglyceride transport protein gene (MTP) variant that improves the patient's response to lomitapide treatment compared to patients that do not express the MTP variant.
10. The method of any one of embodiments 1-9, wherein the first dosing period is at least two weeks.
11. The method of any one of embodiments 1-10, wherein the second dosing period is at least four weeks.
12. The method of any one of embodiments 1-11, wherein the third dosing period is at least four weeks.
13. The method of any one of embodiments 1-12, wherein if the patient's liver aminotransferase (ALT/AST) levels are >5 times the upper limit of normal (ULN) after the first dosing period, the second dosing period or the third dosing period, the patient is withdrawn from lomitapide treatment.
14. The method of embodiment 13, further comprising determining the patient's alkaline phosphatase, total bilirubin and INR.
15. The method of embodiment 14, further comprising reducing the patient's dose to the last dose that provided patient ALT/AST levels of <3 times the ULN.
16. The method of embodiment 15, wherein the patient's dose is reduced from 10 mg to 5 mg.
17. The method of embodiment 15, wherein the patient's dose is reduced from 20 mg to 10 mg.
18. The method of embodiment 15, wherein the patient's dose is reduced from 20 mg to 5 mg.
19. The method of any one of embodiments 1-12, wherein if the patient's ALT/AST levels are from 3-5 times the ULN, confirming the patient's ALT/AST levels are 3-5 times the ULN within one week of the elevated ALT/AST test result.
20. The method of embodiment 19, wherein if the elevated ALT/AST test result is confirmed, determining the patient's alkaline phosphatase, total bilirubin and INR.
21. The method of embodiment 20, further comprising weekly testing the patient's ALT/AST levels, alkaline phosphatase, total bilirubin and INR.
22. The method of embodiment 21, wherein if the patient's total bilirubin and INR increase, ALT/AST levels increase to >5 times ULN or the patient's ALT/AST levels do not fall below <3 times ULN within about 4 weeks, withdrawing the patient from lomitapide treatment.
23. The method of embodiment 22, further comprising reducing the patient's dose to the last dose that provided patient ALT/AST levels of <3 times the ULN.
24. The method of embodiment 23, wherein the patient's dose is reduced from 10 mg to 5 mg.
25. The method of embodiment 23, wherein the patient's dose is reduced from 20 mg to 10 mg.
26. The method of embodiment 23, wherein the patient's dose is reduced from 20 mg to 5 mg.
27. The method any one of embodiments 1-26, further comprising adjusting the patient's daily lomitapide dose to provide fasting triglyceride levels <1000 mg/dL and ALT/AST levels ≤3 times the ULN.
28. The method of embodiment 27, wherein the patient's daily lomitapide dose is increased or decreased from every 2-4 weeks to provide fasting triglyceride levels <1000 mg/dL and ALT/AST levels <3 times the ULN.
29. The method of embodiment 28, wherein the daily dose that provides fasting triglyceride levels <1000 mg/dL and ALT/AST levels ≤3 times the ULN is selected from the group consisting of 5 mg, 10 mg, 20 mg, 30 mg, and 60 mg.
30. The method of any one of embodiments 27-29, wherein the daily dose that provides fasting triglyceride levels ≤1000 mg/dL and ALT/AST levels ≤3 times the ULN is administered for at least 8 weeks.
31. The method of any one of embodiments 1-30, wherein the patient's change in hepatic fat liver is measured during the treatment period.
32. The method of any one of embodiments 1-31, wherein the lomitapide administration does not provide a clinically significant increase in hepatic fat liver during the treatment period.
33. The method of any one of embodiments 1-32, wherein the lomitapide administration substantially decreases the episodes of pancreatitis compared to prior to said treatment.
34. A method of treating familial chylomicronemia syndrome (FCS) in a pediatric patient in need thereof, the method comprising:
   a) orally administering a first daily dose of about 2 mg to about 5 mg of lomitapide, or a pharmaceutically acceptable salt thereof, to the patient for a first dosing period;
   b) measuring the fasting triglyceride levels of the patient after the first dosing period, wherein if the patient's fasting triglyceride levels are ≤1000 mg/dL, the patient is maintained at a daily dose of lomitapide, or a pharmaceutically acceptable salt thereof, of about 2 mg to about 5 mg;
   c) if the patient's measured fasting triglyceride levels are >1000 mg/dL after the first dosing period, the patient is orally administered a second daily dose of about 5 mg to about 10 mg of lomitapide, or a pharmaceutically acceptable salt thereof, for a second dosing period;
   d) measuring the fasting triglyceride levels of the patient after the second dosing period, wherein if the patient's fasting triglyceride levels are ≤1000 mg/dL, the patient is maintained at a daily dose of lomitapide, or a pharmaceutically acceptable salt thereof, of about 5 mg to about 10 mg;
   e) if the patient's measured fasting triglyceride levels are >1000 mg/dL after the second dosing period, the patient is orally administered a third daily dose of about 10 mg to about 20 mg of lomitapide, or a pharmaceutically acceptable salt thereof, for a third dosing period.
35. The method of embodiment 34, wherein the patient's age is from 5 to 10 years.
36. The method of embodiment 35, wherein the daily dose of lomitapide in the first dosing period is 2 mg, the daily dose of lomitapide in the second dosing period is 5 mg, and the daily dose of lomitapide in the third dosing period is 10 mg.
37. The method of embodiment 36, wherein the first dosing period is about 8 weeks, the second dosing period is about 4 weeks, and the third dosing period is about 4 weeks.
38. The method of embodiment 34, wherein the patient's age is from 11 to 15 years.
39. The method of embodiment 38, wherein the daily dose of lomitapide in the first dosing period is 2 mg, the daily dose of lomitapide in the second dosing period is 5 mg, and the daily dose of lomitapide in the third dosing period is 10 mg.
40. The method of embodiment 39, wherein the first dosing period is about 4 weeks, the second dosing period is about 4 weeks, and the third dosing period is about 4 weeks.
41. The method of embodiment 34, wherein the patient's age is 16 to 17 years.
42. The method of embodiment 41, wherein the daily dose of lomitapide in the first dosing period is 5 mg, the daily dose of lomitapide in the second dosing period is 10 mg, and the daily dose of lomitapide in the third dosing period is 20 mg.
43. The method of embodiment 42, wherein the first dosing period is about 4 weeks, the second dosing period is about 4 weeks, and the third dosing period is about 4.

What is claimed:

1. A method of treating familial chylomicronemia syndrome (FCS) in a patient in need thereof, the method comprising:
a) orally administering a first daily dose of about 5 mg of lomitapide, or a pharmaceutically acceptable salt thereof, to the patient for a first dosing period;
b) measuring the fasting triglyceride levels of the patient after the first dosing period, wherein if the patient's fasting triglyceride levels are ≤1000 mg/dL while adhering to a low-fat diet, the patient is maintained at a daily dose of lomitapide, or a pharmaceutically acceptable salt thereof, of about 5 mg;
c) if the patient's measured fasting triglyceride levels are >1000 mg/dL while adhering to a low-fat diet after the first dosing period, the patient is orally administered a second daily dose of about 10 mg of lomitapide, or a pharmaceutically acceptable salt thereof, for a second dosing period;
d) measuring the fasting triglyceride levels of the patient after the second dosing period, wherein if the patient's fasting triglyceride levels are ≤1000 mg/dL while adhering to a low-fat diet, the patient is maintained at a daily dose of lomitapide, or a pharmaceutically acceptable salt thereof, of about 10 mg;
e) if the patient's measured fasting triglyceride levels are >1000 mg/dL while adhering to a low-fat diet after the second dosing period, the patient is orally administered a third daily dose of about 15 mg to about 20 mg of lomitapide, or a pharmaceutically acceptable salt thereof, for a third dosing period.

2. The method of claim 1, wherein the patient is a confirmed homozygote, compound heterozygote or double heterozygote for one or more loss-of-function mutations in genes causing FCS.

3. The method of any one of claims 1-2, wherein the patient has a history of pancreatitis.

4. The method of any one of claim 1-3, wherein the patient's post-heparin plasma lipoprotein lipase (LpL) activity is ≤20% of normal.

5. The method of any one of claim 1-4, wherein the patient has confirmed presence of LpL inactivating antibodies.

6. The method of any one of claim 1-5, wherein the patient's FCS is refractory to plasma LDL apheresis.

7. The method of any one of claim 1-6, wherein the lomitapide is administered as an adjunct to a low-fat diet and other lipid-lowering treatments (such as, statin, ezetimibe, nicotinic acid, bile acid sequestrant, fibrate or LDL apheresis).

8. The method of any one of claims 1-7, wherein the low-fat diet comprises a diet wherein less than 10% of patient's total calories are from fat.

9. The method of any one of claim 1-8, wherein the patient expresses a microsomal triglyceride transport protein gene (MTP) variant that improves the patient's response to lomitapide treatment compared to patients that do not express the MTP variant.

10. The method of any one of claims 1-9, wherein the first dosing period is at least two weeks.

11. The method of any one of claims 1-10, wherein the second dosing period is at least four weeks.

12. The method of any one of claims 1-11, wherein the third dosing period is at least four weeks.

13. The method of any one of claims 1-12, wherein if the patient's liver aminotransferase (ALT/AST) levels are >5 times the upper limit of normal (ULN) after the first dosing period, the second dosing period or the third dosing period, the patient is withdrawn from lomitapide treatment.

14. The method of claim 13, further comprising determining the patient's alkaline phosphatase, total bilirubin and INR.

15. The method of claim 14, further comprising reducing the patient's dose to the last dose that provided patient ALT/AST levels of <3 times the ULN.

16. The method of claim 15, wherein the patient's dose is reduced from 10 mg to 5 mg.

17. The method of claim 15, wherein the patient's dose is reduced from 20 mg to 10 mg.

18. The method of claim 15, wherein the patient's dose is reduced from 20 mg to 5 mg.

19. The method of any one of claims 1-12, wherein if the patient's ALT/AST levels are from 3-5 times the ULN, confirming the patient's ALT/AST levels are 3-5 times the ULN within one week of the elevated ALT/AST test result.

20. The method of claim 19, wherein if the elevated ALT/AST test result is confirmed, determining the patient's alkaline phosphatase, total bilirubin and INR.

21. The method of claim 20, further comprising weekly testing the patient's ALT/AST levels, alkaline phosphatase, total bilirubin and INR.

22. The method of claim 21, wherein if the patient's total bilirubin and INR increase, ALT/AST levels increase to >5 times ULN or the patient's ALT/AST levels do not fall below <3 times ULN within about 4 weeks, withdrawing the patient from lomitapide treatment.

23. The method of claim 22, further comprising reducing the patient's dose to the last dose that provided patient ALT/AST levels of <3 times the ULN.

24. The method of claim 23, wherein the patient's dose is reduced from 10 mg to 5 mg.

25. The method of claim 23, wherein the patient's dose is reduced from 20 mg to 10 mg.

26. The method of claim 23, wherein the patient's dose is reduced from 20 mg to 5 mg.

27. The method any one of claims 1-26, further comprising adjusting the patient's daily lomitapide dose to provide fasting triglyceride levels ≤1000 mg/dL and ALT/AST levels ≤3 times the ULN.

28. The method of claim 27, wherein the patient's daily lomitapide dose is increased or decreased from every 2-4 weeks to provide fasting triglyceride levels ≤1000 mg/dL and ALT/AST levels ≤3 times the ULN.

29. The method of claim 28, wherein the daily dose that provides fasting triglyceride levels ≤1000 mg/dL and ALT/AST levels ≤3 times the ULN is selected from the group consisting of 5 mg, 10 mg, 15 mg, 20 mg, 30 mg, 40 mg, 50 mg, and 60 mg.

30. The method of any one of claims 27-29, wherein the daily dose that provides fasting triglyceride levels ≤1000 mg/dL and ALT/AST levels ≤3 times the ULN is administered for at least 8 weeks.

31. The method of any one of claims 1-30, wherein the patient's change in hepatic fat liver is measured during the treatment period.

32. The method of any one of claims 1-31, wherein the lomitapide administration does not provide a clinically significant increase in hepatic fat liver during the treatment period.

33. The method of any one of claims 1-32, wherein the lomitapide administration substantially decreases the episodes of pancreatitis compared to prior to said treatment.

34. A method of treating familial chylomicronemia syndrome (FCS) in a pediatric patient in need thereof, the method comprising:
a) orally administering a first daily dose of about 2 mg to about 5 mg of lomitapide, or a pharmaceutically acceptable salt thereof, to the patient for a first dosing period;
b) measuring the fasting triglyceride levels of the patient after the first dosing period, wherein if the patient's fasting triglyceride levels are ≤1000 mg/dL, the patient is maintained at a daily dose of lomitapide, or a pharmaceutically acceptable salt thereof, of about 2 mg to about 5 mg;
c) if the patient's measured fasting triglyceride levels are >1000 mg/dL after the first dosing period, the patient is orally administered a second daily dose of about 5 mg to about 10 mg of lomitapide, or a pharmaceutically acceptable salt thereof, for a second dosing period;
d) measuring the fasting triglyceride levels of the patient after the second dosing period, wherein if the patient's fasting triglyceride levels are ≤1000 mg/dL, the patient is maintained at a daily dose of lomitapide, or a pharmaceutically acceptable salt thereof, of about 5 mg to about 10 mg;
e) if the patient's measured fasting triglyceride levels are >1000 mg/dL after the second dosing period, the patient is orally administered a third daily dose of about 10 mg to about 20 mg of lomitapide, or a pharmaceutically acceptable salt thereof, for a third dosing period.

35. The method of claim 34, wherein the patient's age is from 5 to 10 years.

36. The method of claim 35, wherein the daily dose of lomitapide in the first dosing period is 2 mg, the daily dose of lomitapide in the second dosing period is 5 mg, and the daily dose of lomitapide in the third dosing period is 10 mg.

37. The method of claim 36, wherein the first dosing period is about 8 weeks, the second dosing period is about 4 weeks, and the third dosing period is about 4 weeks.

38. The method of claim 34, wherein the patient's age is from 11 to 15 years.

39. The method of claim 38, wherein the daily dose of lomitapide in the first dosing period is 2 mg, the daily dose of lomitapide in the second dosing period is 5 mg, and the daily dose of lomitapide in the third dosing period is 10 mg.

40. The method of claim 39, wherein the first dosing period is about 4 weeks, the second dosing period is about 4 weeks, and the third dosing period is about 4 weeks.

41. The method of claim 34, wherein the patient's age is 16 to 17 years.

42. The method of claim 41, wherein the daily dose of lomitapide in the first dosing period is 5 mg, the daily dose of lomitapide in the second dosing period is 10 mg, and the daily dose of lomitapide in the third dosing period is 20 mg.

43. The method of claim 42, wherein the first dosing period is about 4 weeks, the second dosing period is about 4 weeks, and the third dosing period is about 4.
